SEQUENCE LISTING (7 sequences)

SEQ ID NO: 1 - protein, 15 aa, mammalian
  MOD_RES (7)..(8): Gly-Leu phosphorus mimic
GPPGPQGLAGQRGIV

SEQ ID NO: 2 - protein, 15 aa, mammalian
  MOD_RES (4)..(5): Gly-Val phosphorus mimic
GPPGVVGEQGEQGPP

SEQ ID NO: 3 - protein, 12 aa, mammalian
  MOD_RES (4)..(5): Gly-Leu phosphorus mimic
GPPGLLGLQGLP

SEQ ID NO: 4 - protein, 12 aa, mammalian
  MOD_RES (4)..(5): Gly-Leu phosphorus mimic
GPAGLPGSMGPP

SEQ ID NO: 5 - protein, 21 aa, mammalian
  MOD_RES (3)..(21): x= hydroxyproline
GPHGPPGVVGEQGEQGPPGPH

SEQ ID NO: 6 - protein, 18 aa, mammalian
GPHGPQGLAGQRGIRGPH

SEQ ID NO: 7 - protein, 15 aa, mammalian
GTLGELEGRGTLGIS

(19) United States
(12) Patent Application Publication
(10) Pub. No.: US 2008/0125354 A1
(43) Pub. Date: May 29, 2008

(54) SELECTIVE INHIBITION OF MATRIX METALLOPROTEINASES

(75) Inventors: Gregg B. Fields, Delray Beach, FL (US); Robert Hammer, Baton Rouge, LA (US)

Correspondence Address: AKERMAN SENTERFITT, P.O. BOX 3188, WEST PALM BEACH, FL, 33402-3188, US

(73) Assignees: Florida Atlantic University, Boca Raton, FL (US); Board of Supervisors of Louisiana State University and Agricultural and Mechanical College, Baton Rouge, LA (US)

(21) Appl. No.: 11/562,295

(22) Filed: Nov. 21, 2006

Related U.S. Application Data
(60) Provisional application No. 60/738,616, filed on Nov. 21, 2005.

(51) Int. Cl. A61K 38/02; A61P 21/00; A61K 31/664; A61K 31/662; A61P 43/00

(52) U.S. Cl. 514/7; 514/137; 514/141; 514/114

(57) ABSTRACT

Synthetic triple-helical peptides (THPs) are used for the design and synthesis of triple-helical transition state analog inhibitors. These inhibitors feature a phosphonate ester or phosphinic moiety in place of the scissle bond. These groups inhibit MMPs, and methods been developed for their convenient incorporation within a peptide sequence by solid-phase methods.

### CROSS REFERENCE TO RELATED APPLICATIONS

The present application claims the priority of U.S. provisional patent application No. 60/738,616, entitled “SELECTIVE INHIBITION OF MATRIX METALLOPROTEINASES,” filed Nov. 21, 2005, which is incorporated herein by reference in its entirety.

### STATEMENT AS TO FEDERALLY FUNDED RESEARCH

This invention was made with United States government support under grant numbers 1R01-CA98799 awardedbyNational Institutes of Health/National Cancer Institute. The United States government may have certain rights in the invention.

### FIELD OF THE INVETION

The invention is related to matrix metalloproteinase inhibitors and treatment of diseases associated with collagen breakdown.

### BACKGROUND

The triple-helical conformation of collagen has long been recognized for its role in structural stabilization of connective tissue. The dissolution of the collagen triple-helix has thus been implicated in a variety of diseases, such as arthritis, that affect the structural integrity of various components of the body. Collagen also provides a barrier between tissues and cells; destruction of this barrier plays a role in tumor cell invasion and the metastatic process. A family of metalloenzymes, the matrix metalloproteinases (MMPs), has been recognized for their ability to hydrolyze collagen (“collagenolytic” activity). The MMP family has thus been the subject of intense research efforts, in order to elucidate their mechanisms of action, and allow for rational design of effective and selective MMP inhibitors. We have previously developed novel methodology for constructing synthetic triple-helical peptides (THPs) and have now applied these synthetic proteins for the design and synthesis of triple-helical and poly-proline type II transition state analog inhibitors. These inhibitors feature a phosphinic moiety in place of the scissle bond. This group have been shown previously to inhibit MMPs, but only recently have the methods been developed for its convenient incorporation within a peptide sequence by solid-phase methods.

### SUMMARY

Dysregulation of matrix metalloproteinase (MMP) activity has been implicated in primary and metastatic tumor growth, angiogenesis, and uncontrolled degradation of ECM components. Since the hydrolysis of collagen is one of the committed steps in ECM turnover, we have investigated ways of modulating collagenolytic activity by creating selective MMP inhibitors. Enzyme inhibition is based upon a pseudo-peptide bond derived from phosphinic acid. The phosphinic acid group acts as a tetrahedral transition-state analog, which mimics the water-bound peptide bond of a protein substrate during hydrolysis. Our first inhibitor design utilized a triple-helical collagen model peptide substrate hydrolyzed selectively by the gelatinases (MMP-2 and -9) [J. Biol. Chem. 278, 18140-18145 (2003)] which is incorporated by reference herein, in its entirety. The P₁-P₁′ subsites of the triple-helical peptide, which incorporate Gly-Val in the substrate, were substituted by a phosphinic acid pseudo-dipeptide. This modification of the peptide backbone would result in binding of the triple-helical peptide to the enzyme active site, but not hydrolysis. Studies revealed Kᵢ values of 5.48 and 2.20 nM for MMP-2 and MMP-9, respectively, and IC₅₀ values in the low to middle micromolar range for MMP-8 and MMP-13. Neither MMP-14, MMP-1 nor MMP-3 were inhibited. The result of this first generation design for a pseudo-peptide inhibitor possessing triple-helical structure is a compound with high affinity and selectivity for the gelatinases. Our second inhibitor design utilized a triple-helical collagen model peptide substrate hydrolyzed by collagenases (MMP-1, -2, -8, -13, and -14) [Biochemistry 43, 11474-11481 (2004)] which is incorporated by reference herein, in its entirety. The P₁-P₁′ subsites of the triple-helical peptide, which incorporate Gly-Leu in the substrate, were substituted by a phosphinic acid pseudo-dipeptide. Studies revealed Kᵢ values of 18.6, 0.40, and 0.12 nM for MMP-1, MMP-2, and MMP-9, respectively. We anticipate that other substitutions in the P and P′ subsites of triple-helical peptides, along with modulation of triple-helix stability, can be applied to create additional selective pseudo-peptide MMP inhibitors.

In another preferred embodiment, the phosphonamide is of the general formula: Ψ(PO₂H—NH) wherein Ψ=NH; the phosphinic peptide is of the general formula: Ψ(PO₂H—CH₂) wherein Ψ=CH₂; the phosphonate ester is of the general formula: Ψ{PO₂H-Q}; Ψ=O.

In another preferred embodiment the matrix metalloproteinase inhibitor comprises any one or more of SEQ ID NOS: 1-6 as well as the aggrecanase substrate sequence Gly-Thr-Lys(Mca)-Gly-Glu-Leu-Glu-Gly-Arg-Gly-Thr-Lys(Dnp)-Gly-Ile-Ser. (SEQ ID NO: 7). Preferably the inhibitor comprises at least one or more Gly-Pro-Hyp and Gly-Pro-Flp sequences. The inhibitor may also comprises at least one or more Gly-Pro-Hyp or Gly-Pro-Flp sequences. Preferably, the Gly-Pro-Flp is at the N-terminus and/or C-terminus of the inhibitor. Preferably, the inhibitor comprises a plurality of Gly-Pro-Hyp sequences; preferably, the inhibitor comprises between about one to ten Gly-Pro-Hyp sequences.

In another preferred embodiments, the matrix metalloproteinase inhibitor P and P′ subsites are substituted wherein the substitutions comprise phosphinate, phosphonate ester or phosphoramide mimics with Gly or Ala in the P₁ subsite and/or Cys(Mob) in the P₁′ subsite. In addition, the P₂ subsite may accommodate ornithine (Om) while the P₂′ and/or P₃′ subsite may accommodate Glu.

In another preferred embodiment, a pharmaceutical composition comprises (R,S)-2-Isopropyl-3-((1-(N-(9-Fluorenylmethoxycarbonyl)amino)-methyl)-adamantyloxyphosphinyl) propanoic acid.

In another preferred embodiment, (R,S)-2-Isopropyl-3-((1-(N-(9-Fluorenylmethoxycarbonyl)amino)-methyl)-adamantyloxyphosphinyl) propanoic acid further comprises substituted P, P′, P₂′ and P₃′ subsites. Preferably, the P and P′ subsites are substituted with molecules comprising phosphinate, phosphonate ester or phosphoramide mimics with Gly or Ala in the P₁ subsite and/or Cys(Mob) in the P₁′ subsite; Ornithine (Om) in the P₂ subsite; and, Glu in the P₂′ and/or P₃′ subsite.

In another preferred embodiment, a method of treating patients suffering from metalloproteinase mediated disease condition comprises: administering to a patient in need thereof, a pharmaceutical composition comprising a matrix metalloproteinase inhibitor wherein the inhibitor is a triple helix phosphorus based inhibitor comprising a phosphonamide, phosphinic peptide or phosphonate ester.

In another preferred embodiment, the method of treating patients suffering from metalloproteinase mediated disease condition comprises administering a pharmaceutical composition comprising a matrix metalloproteinase inhibitor wherein the inhibitor comprises any one or more of SEQ ID NOS: 1-6.

In a preferred embodiment, the inhibitor administered to patients suffering from metalloproteinase mediated disease condition, the inhibitor comprises at least one or more Gly-Pro-Hyp and/or Gly-Pro-Flp sequences.

The present invention also relates to a pharmaceutical composition for the treatment of a condition comprising any one or more of: arthritis, cancer, synergy with cytotoxic anticancer agents, tissue ulceration, macular degeneration, restenosis, periodontal disease, epidermolysis bullosa, scieritis, in combination with standard NSAID'S and analgesics and other diseases characterized by matrix metalloproteinase activity, AIDS, sepsis, septic shock and other metalloprotease mediated diseases in a mammal, including a human, comprising an amount of the inventive compounds, e.g. SEQ ID NOS: 1-7 or a pharmaceutically acceptable salt thereof effective in such treatments and a pharmaceutically acceptable carrier.

The present invention also relates to a method for the inhibition of (a) matrix metalloproteinases in a mammal, including a human, comprising administering to said mammal an effective amount of the compounds of the invention or a pharmaceutically acceptable salt thereof.

The present invention also relates to a method for the treatment of a condition comprising any one or more of: arthritis, cancer, synergy with cytotoxic anticancer agents, tissue ulceration, macular degeneration, restenosis, periodontal disease, epidermolysis bullosa, scleritis, in combination with standard NSAID'S and analgesics and other diseases characterized by matrix metalloproteinase activity, AIDS, sepsis, septic shock and other metalloprotease mediated diseases in a mammal, including a human, comprising an amount of the inventive compounds, e.g. SEQ ID NOS: 1-7 or a pharmaceutically acceptable salt thereof effective in such treatments and a pharmaceutically acceptable carrier.

Other aspects of the invention are described infra.

### BRIEF DESCRIPTION OF THE DRAWINGS

The invention is pointed out with particularity in the appended claims. The above and further advantages of this invention may be better understood by referring to the following description taken in conjunction with the accompanying drawings, in which:

FIG. 1 is a schematic representation showing the synthesis of a phosphinate dipeptide mimic.

FIG. 2 is a schematic showing the preparation of the phosphonate dipeptide analog.

FIG. 3A is a schematic representation showing tetrahedral intermediate (boxed) and statine and phosphorus-based transition state analog inhibitors. FIG. 3B is a schematic illustration showing phosphonate 5 and its thioanalog 6.

FIG. 4 is a schematic representation showing a typical phosphorus or thiophosphorus triple-helical substrate analog to be prepared by solid-phase peptide synthesis.

FIG. 5A is a plot showing RP-HPLC analysis of phosphinate collagen mimic without C₆ tail. FIG. 5B is a plot showing RP-HPLC analysis of phosphinate collagen mimic with C₆ tail.

FIG. 6 is a graph showing CD wavelength scans of f1-f4.

FIG. 7 is a graph showing CD temperature scans of f1-f4.

FIG. 8 is a graph showing inhibition of MMP-2 by peptide-amphiphile 1.

FIG. 9 is a graph showing inhibition of MMP-2 by peptide-amphiphile 2.

FIG. 10 is a graph showing inhibition of MMP-8 by f1, f2, f3, and f4.

### DETAILED DESCRIPTION

The invention provides compositions of MMP inhibitors and methods of treatment of a matrix metalloproteinase mediated disease conditions such as arthritis, cancer and the like, wound healing and tissue regeneration. The triple-helical conformation of collagen has long been recognized for its role in structural stabilization of connective tissue. The dissolution of the collagen triple-helix has thus been implicated in a variety of diseases, such as arthritis, that affect the structural integrity of various components of the body. Collagen also provides a barrier between tissues and cells; destruction of this barrier plays a role in tumor cell invasion and the metastatic process. A family of metalloenzymes, the matrix metalloproteinases (MMPs), has been recognized for their ability to hydrolyze collagen (“collagenolytic” activity). We have previously developed novel methodology for constructing synthetic triple-helical peptides (THPs) and have now applied these synthetic proteins for the design and synthesis of triple-helical and poly-proline type II transition state analog inhibitors. These inhibitors feature a phosphinic moiety in place of the scissle bond. This group inhibits MMPs, and methods have been developed for its convenient incorporation within a peptide sequence by solid-phase methods.

### DEFINITIONS

The term “inhibitor” means a compound of this invention that inhibits the function of a metalloproteinase.

“Prodrugs” are intended to include any covalently bonded carriers which release an active parent drug of the present invention in vivo when such prodrug is administered to a mammalian subject.

As used herein, an “MP related disorder” or “MP related disease” is one that involves unwanted or elevated MP activity in the biological manifestation of the disease or disorder; in the biological cascade leading to the disorder; or as a symptom of the disorder, e.g. cancer, metastatic cancer, tissue ulceration, macular degeneration, restenosis, periodontal disease, epidermolysis bullosa, scleritis, osetoarthritis, and the like. This “involvement” of the MP includes: 1. The unwanted or elevated MP activity as a “cause” of the disorder or biological manifestation, whether the activity is elevated genetically, by infection, by autoimmunity, trauma, biomechanical causes, lifestyle [e.g. obesity] or by some other cause; 2. The MP as part of the observable manifestation of the disease or disorder. That is, the disease or disorder is measurable in terms of the increased MP activity. From a clinical standpoint, unwanted or elevated MP levels indicate the disease; however, MPs need not be the “hallmark” of the disease or disorder; or 3. The unwanted or elevated MP activity is part of the biochemical or cellular cascade that results or relates to the disease or disorder. In this respect, inhibition of the MP activity interrupts the cascade, and thus controls the disease.

As used herein, a “disorder associated with excess or undesired metalloprotease activity” is any disorder characterized by degradation of matrix proteins. The methods of the invention are useful in treating disorders described above.

The term “treatment” is used herein to mean that, at a minimum, administration of a compound of the present invention mitigates a disease associated with unwanted or elevated MP activity in a mammalian subject, preferably in humans. Thus, the term “treatment” includes: preventing an MP-mediated disease from occurring in a mammal, particularly when the mammal is predisposed to acquiring the disease, but has not yet been diagnosed with the disease; inhibiting the MP-mediated disease; and/or alleviating the MP-mediated disease. Insofar as the methods of the present invention are directed to preventing disease states associated with unwanted MP activity, it is understood that the term “prevent” does not require that the disease state be completely thwarted. (See Webster's Ninth Collegiate Dictionary.) Rather, as used herein, the term preventing refers to the ability of the skilled artisan to identify a population that is susceptible to MP-related disorders, such that administration of the compounds of the present invention may occur prior to onset of the disease. The term does not imply that the disease state be completely avoided. For example, osteoarthritis (OA) is the most common rheumatological disease with some joint changes radiologically detectable in 80% of people over 55 years of age. Fife, R. S., “A Short History of Osteoarthritis”, Osteoarthritis: Diagnosis and Medical/Surgical Management, R. W. Moskowitz, D. S. Howell, V. M. Goldberg and H. J. Mankin Eds., p 11-14 (1992). A common risk factor that increases the incidence of OA is traumatic injury of the joint. Surgical removal of the meniscus following knee injury increases the risk of radiographically detectable OA and this risk increases with time. Roos, H et al. “Knee Osteoarthritis After Menisectomy: Prevalence of Radiographic Changes After Twenty-one Years, Compared with Matched Controls.” Arthritis Rheum., Vol. 41, pp 687-693; Roos, H et al. “Osteoarthritis of the Knee After Injury to the Anterior Cruciate Ligament or Meniscus: The Influence of Time and Age.” Osteoarthritis Cartilege, Vol. 3, pp 261-267 (1995). Thus, this patient population is identifiable and could receive administration of a compound of the present invention before progression of the disease. Thus, progression of OA in such individuals would be “prevented”.

Metalloprotease Inhibitors: The inhibitor design first utilized a triple-helical collagen model peptide substrate hydrolyzed selectively by the gelatinases (MMP-2 and -9). The P₁-P₁′ subsites of the triple-helical peptide, which incorporate Gly-Val in the substrate, were substituted by a phosphinic acid pseudo-dipeptide. This modification of the peptide backbone should result in binding of the triple-helical peptide to the enzyme active site, but not hydrolysis. Studies revealed Kᵢ values of 5.48 and 2.20 nM for MMP-2 and MMP-9, respectively, and IC₅₀ values in the low to middle micromolar range for MMP-8 and MMP-13. Neither MMP-14, MMP-1 nor MMP-3 were inhibited. The result of this first generation design for a pseudo-peptide inhibitor possessing triple-helical structure is a compound with high affinity and selectivity for the gelatinases. The second inhibitor design utilized a triple-helical collagen model peptide substrate hydrolyzed by collagenases (MMP-1, -2, -8, -13, and -14). The P₁-P₁′ subsites of the triple-helical peptide, which incorporate Gly-Leu in the substrate, were substituted by a phosphinic acid pseudo-dipeptide. Studies revealed Kᵢ values of 18.6, 0.40, and 0.12 nM for MMP-1, MMP-2, and MMP-9, respectively. We anticipate that other substitutions in the P and P′ subsites of triple-helical peptides, along with modulation of triple-helix stability, can be applied to create additional selective pseudo-peptide MMP inhibitors.

Collagen Catabolism by Matrix Metalloproteinases: Catabolism of extracellular matrix (ECM) components has been ascribed to a family of Zn²⁺ metalloenzymes. These matrix metalloproteinases (MMPs; also termed matrixins) are believed to be important in connective tissue remodeling during development and wound healing. MMPs have also been implicated in a variety of disease states, including arthritis, glomerulonephritis, periodontal disease, tissue ulcerations and tumor cell invasion and metastasis. Amongst the roles of MMPs in metastasis are primary and metastatic tumor growth, angiogenesis, and degradation of basement membrane barriers during tumor cell invasion. Because of their involvement in pathological conditions, it is desirable to design inhibitors of MMP family members.

There are at least 25 members of the MMP family, categorized based on their domain structures and their preferences for macromolecular substrates (Nelson, A. et al., (2000) J. Clin. Oncol. 18, 1135-1149., Woessner, J. F., and Nagase, H. (2000) Matrix Metalloproteinases and TIMPs, Oxford University Press, Oxford). Most MMPs contain a propeptide domain, a catalytic domain, and a hemopexin/vitronectin-like domain (Woessner, J. F., and Nagase, H., supra). The MMP family includes MMP-1 (interstitial cotlagenase, collagenase 1), MMP-2 (gelatinase A), MMP-3 (stromelysin 1), MMP-7 (pump 1, matrilysin), MMP-8 (neutrophil collagenase, collagenase 2), MMP-9 (gelatinase B), MMP-10 (stromelysin 2), MMP-11 (stromelysin 3), MMP-12 (metalloelastase, macrophage elastase), MMP-13 (collagenase 3), five membrane-type MMPs (MT-MMPs) (MMP-14, MMP-15, MMP-16, MMP-17, MMP-21), MMP-18 (Xenopus collagenase 4), MMP-19, MMP-20 (enamelysin), MMP-22 (chicken CMMP), MMP-23, MMP-24, MMP-25, MMP-26 (endometase), MMP-27, and MMP-28 (epilysin). Some redundancy of MMP family member numbering exists: telopeptidase, later designated MMP-4, and 3/4-collagenase (MMP-5) are MMP-3 and MMP-2, respectively; MMP-6 (acid metalloproteinase) was shown to be MMP-3.

A number of MMP family members possess collagenolytic activity, which is one of the “committed” steps in ECM turnover. For example, interstitial collagens (types I-III) are hydrolyzed by MMP-1, MMP-2, MMP-8, MMP-13, MMP-14, and MMP-18. Conversely, MMP-3 and MMP-9 bind to type I collagen, but do not cleave the triple-helical domain. (Allan, J. A., et al. (1991) J. Cell Sci. 99, 789-795; Murphy, G., et al. (1992) J. Biol. Chem. 267, 9612-9618; Allan, J. A., et al. (1995) Biochem. J. 309, 299-306.18-20).

Collagen-Model Triple-Helical Peptides: One approach to better understand the mechanisms of collagenolytic activity is to use models of collagen cleavage sites. In order to be successful, triple-helical peptide (THP) substrates would be required to (a) incorporate a sequence that could be cleaved in triple-helical conformation and (b) have sufficient thermal stability to remain triple-helical under assay conditions. The interstitial collagen sequences targeted by MMP-1, MMP-2, MMP-8, and MMP-13 have been identified, and a model collagenase cleavage site has been proposed based on the combination of triple-helical collagen primary, secondary, and super-secondary structures. Several methods have been described for the synthesis of THPs incorporating collagen-like sequences and with Tₘ values greater than 30° C. Our laboratory has developed a solid-phase THP synthetic method by which the non-covalent self-assembly of lipophilic molecules, N-terminally linked to a peptide, can be used to form stable triple-helical “peptide-amphiphiles.”

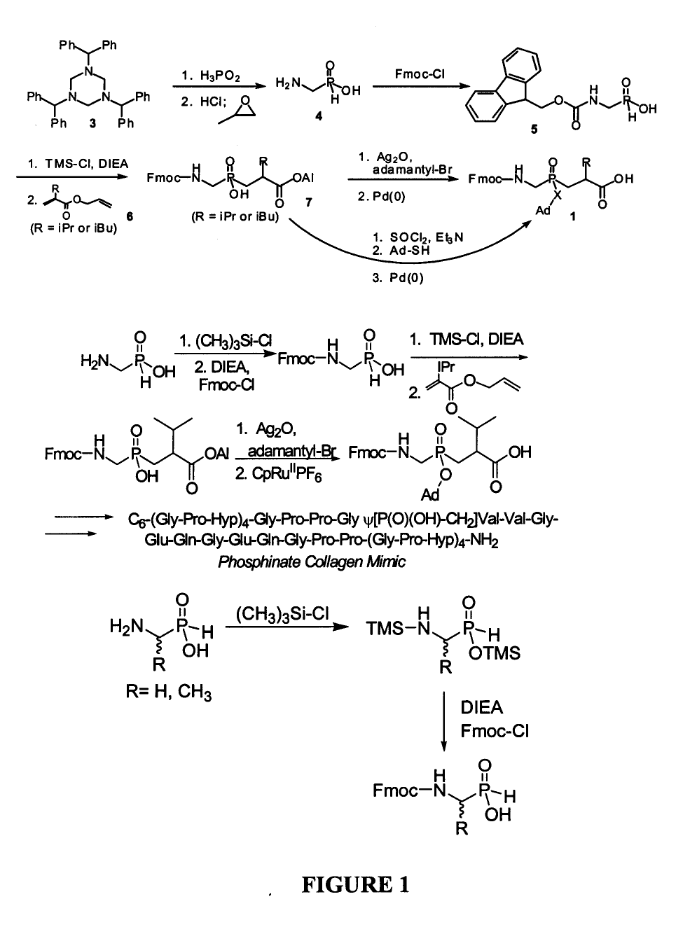

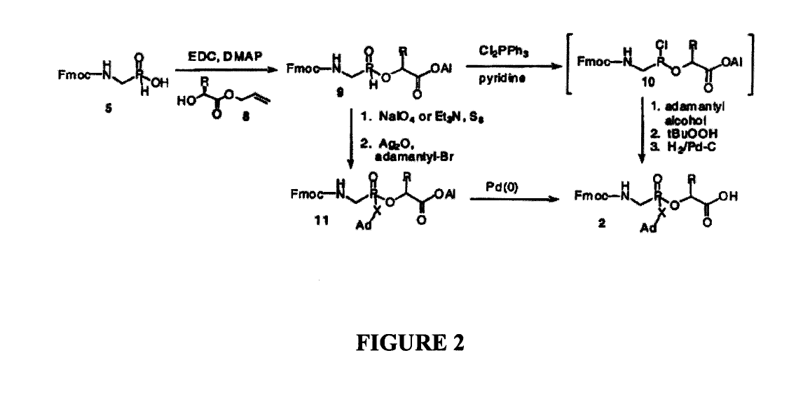

Design of MMP Inhibitors: To date, the vast majority of MMP inhibitors contain a hydroxamic acid group which chelates the active site zinc. However, the hydroxamic acid usually represents a terminal point in the chain, and thus residues that interact with only one side of the enzyme active site can be incorporated. FIG. 1 is a schematic representation showing the synthesis of phosphinate dipeptide mimic. FIG. 2 is a schematic showing the preparation of the phosphonate dipeptide analog.

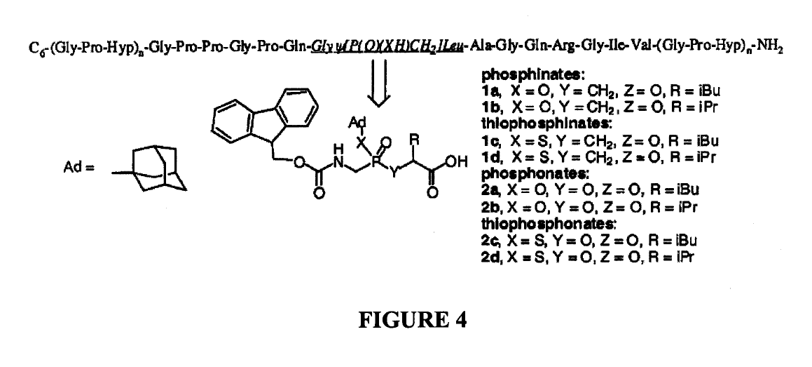

As both phosphinate and phosphonate mimics have been effective at inhibiting MMPs, both phosphinate and phosphonate triple-helical collagen substrate analogs described in Table 1 will be made. Additionally the thiophosphinate and thiophosphonate versions of peptide mimics which show promise as triple-helical inhibitors will be made. The MMP cleavage site in each of the sequences will be replaced by either a Gly-Leu phosphorus mimic or a Gly-Val phosphorus mimic [for α1(V)-436-450 sequence]. FIG. 4 shows the typical sequence that will be prepared using stepwise solid-phase peptide synthesis, where the Gly-Leu or Gly-Val phosphorus analog will be inserted as a phosphorus ester-protected, Fmoc-protected dipeptide 1 or 2.

Pharmaceutical Compositions: pharmaceutical compositions of this invention may be administered in standard manner for the disease condition that it is desired to treat, for example by oral, topical, parenteral, buccal, nasal, vaginal or rectal administration or by inhalation. For these purposes the compounds of this invention may be formulated by means known in the art into the form of, for example, tablets, capsules, aqueous or oily solutions, suspensions, emulsions, creams, ointments, gels, nasal sprays, suppositories, finely divided powders or aerosols for inhalation, and for parenteral use (including intravenous, intramuscular or infusion) sterile aqueous or oily solutions or suspensions or sterile emulsions.

In addition to the compounds of the present invention the pharmaceutical composition of this invention may also contain, or be co-administered (simultaneously or sequentially) with, one or more pharmacological agents of value in treating one or more disease conditions referred to hereinabove.

The pharmaceutical compositions of this invention will normally be administered to humans so that, for example, a daily dose of 0.5 to 75 mg/kg body weight (and preferably of 0.5 to 30 mg/kg body weight) is received. This daily dose may be given in divided doses as necessary, the precise amount of the compound received and the route of administration depending on the weight, age and sex of the patient being treated and on the particular disease condition being treated according to principles known in the art.

Typically unit dosage forms will contain about 1 mg to 500 mg of a compound of this invention.

In yet a further aspect the present invention provides a method of treating a metalloproteinase mediated disease condition which comprises administering to a warm-blooded animal a therapeutically effective amount of a compound of the formula I or a pharmaceutically acceptable salt or in vivo hydrolysable ester thereof. Metalloproteinase mediated disease conditions include arthritis (such as osteoarthritis), atherosclerosis, chronic obstructive pulmonary diseases (COPD).

Some of the invention compounds are capable of further forming nontoxic pharmaceutically acceptable salts, including, but not limited to, acid addition and/or base salts. The acid addition salts are formed from basic invention compounds, whereas the base addition salts are formed from acidic invention compounds. All of these forms are within the scope of the compounds useful in the invention.

Pharmaceutically acceptable acid addition salts of the basic invention compounds include nontoxic salts derived from inorganic acids such as hydrochloric, nitric, phosphoric, sulfuric, hydrobromic, hydroiodic, hydrofluoric, phosphorous, and the like, as well nontoxic salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, malate, tartrate, methanesulfonate, and the like. Also contemplated are salts of amino acids such as arginate and the like and gluconate, galacturonate (see, for example, Berge S. M. et al., “Pharmaceutical Salts,” J. of Pharma. Sci., 1977; 66:1).

An acid addition salt of a basic invention compound is prepared by contacting the free base form of the compound with a sufficient amount of a desired acid to produce a nontoxic salt in the conventional manner. The free base form of the compound may be regenerated by contacting the acid addition salt so formed with a base, and isolating the free base form of the compound in the conventional manner. The free base forms of compounds prepared according to a process of the present invention differ from their respective acid addition salt forms somewhat in certain physical properties such as solubility, crystal structure, hygroscopicity, and the like, but otherwise free base forms of the invention compounds and their respective acid addition salt forms are equivalent for purposes of the present invention.

A nontoxic pharmaceutically acceptable base addition salt of an acidic invention compound may be prepared by contacting the free acid form of the compound with a metal cation such as an alkali or alkaline earth metal cation, or an amine, especially an organic amine. Examples of suitable metal cations include sodium cation (Na⁺), potassium cation (K⁺), magnesium cation (Mg²⁺), calcium cation (Ca²⁺), and the like. Examples of suitable amines are N,N′-dibenzylethylenediamine, chloroprocaine, choline, diethanolamine, dicyclohexylamine, ethylenediamine, N-methylglucamine, and procaine (see, for example, Berge, supra., 1977).

A base addition salt of an acidic invention compound may be prepared by contacting the free acid form of the compound with a sufficient amount of a desired base to produce the salt in the conventional manner. The free acid form of the compound may be regenerated by contacting the salt form so formed with an acid, and isolating the free acid of the compound in the conventional manner. The free acid forms of the invention compounds differ from their respective salt forms somewhat in certain physical properties such as solubility, crystal structure, hygroscopicity, and the like, but otherwise the salts are equivalent to their respective free acid for purposes of the present invention.

Certain invention compounds can exist in unsolvated forms as well as solvated forms, including hydrated forms. In general, the solvated forms, including hydrated forms, are equivalent to unsolvated forms and are encompassed within the scope of the present invention.

Certain of the invention compounds possess one or more chiral centers, and each center may exist in the R or S configuration. An invention compound includes any diastereomeric, enantiomeric, or epimeric form of the compound, as well as mixtures thereof.

The invention compounds also include isotopically-labeled compounds, which are identical to those recited above, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as ²H, .³H, ¹³C, ¹⁴C, ¹⁵N, ¹⁸O, ¹⁷O, ³¹P, ³²P, ³⁵S, ¹⁸F and ³⁶Cl, respectively. Compounds of the present invention and pharmaceutically acceptable salts of said compounds which contain the aforementioned isotopes and/or other isotopes of other atoms are within the scope of this invention. Certain isotopically labeled compounds of the present invention, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances.

Disease Treatments: One of ordinary skill in the art will appreciate that the compounds of the invention are useful in treating a diverse array of diseases. Advantageously, many MPs are not distributed evenly throughout the body. Thus, the distribution of MPs expressed in various tissues are often specific to those tissues. For example, the distribution of metalloproteases implicated in the breakdown of tissues in the joints is not the same as the distribution of metalloproteases found in other tissues. Though not essential for activity or efficacy, certain diseases, disorders, and unwanted conditions preferably are treated with compounds that act on specific MPs found in the affected tissues or regions of the body. For example, a compound which displays a higher degree of affinity and inhibition for an MP found in the joints (e.g. chondrocytes) would be preferred for treatment of a disease, disorder, or unwanted condition found there than other compounds which are less specific. The compounds of the instant invention are shown to be specific and selective. As such, these inhibitors can be used together (e.g. target differing MMP's involved in the disorder), alone or in combinations with other compounds to treat the metalloprotease disorders.

Determination of the specificity of an inhibitor of a specific MP is within the skill of the artisan in that field. The Examples section which follows, provides details as to identification of the specific inhibitors. Furthermore, appropriate assay conditions can be found in the literature. Specifically, assays are known for stromelysin and collagenase. For example, U.S. Pat. No. 4,743,587 references the procedure of Cawston, et al., Anal Biochem (1979) 99:340-345. See also, Knight, C. G. et al., “A Novel Coumarin-Labelled Peptide for Sensitive Continuous Assays of the Matrix Metalloproteases”, FEBS Letters, Vol. 296, pp. 263-266 (1992). The use of a synthetic substrate in an assay is described by Weingarten, H., et al., Biochem Biophy Res Comm (1984) 139:1184-1187. Any standard method for analyzing the breakdown of structural proteins by MPs can, of course, be used. The ability of compounds of the invention to inhibit metalloprotease activity can be tested in the assays found in the literature, or variations thereof. Isolated metalloprotease enzymes can be used to confirm the inhibiting activity of the invention compounds, or crude extracts which contain the range of enzymes capable of tissue breakdown can be used.

The compounds of this invention are also useful for prophylactic or acute treatment. They are administered in any way the skilled artisan in the fields of medicine or pharmacology would desire. It is immediately apparent to the skilled artisan that preferred routes of administration will depend upon the disease state being treated and the dosage form chosen. Preferred routes for systemic administration include administration perorally or parenterally. However, the skilled artisan will readily appreciate the advantage of administering the MP inhibitor directly to the affected area for many diseases, disorders, or unwanted conditions. For example, it may be advantageous to administer MP inhibitors directly to the area of the disease, disorder, or unwanted condition such as in the area affected by surgical trauma (e.g., angioplasty), scarring, burning (e.g., topical to the skin), or for ophthalmic and periodontal indications.

Because the remodeling of bone involves MPs, the compounds of the invention are useful in preventing prosthesis loosening. It is known in the art that over time prostheses loosen, become painful, and may result in further bone injury, thus demanding replacement. The need for replacement of such prostheses includes those such as in joint replacements (for example hip, knee and shoulder replacements), dental prosthesis, including dentures, bridges and prosthesis secured to the maxilla and/or mandible.

MPs are also active in remodeling of the cardiovascular system (for example, in congestive heart failure). It has been suggested that one of the reasons angioplasty has a higher than expected long term failure rate (reclosure over time) is that MP activity is not desired or is elevated in response to what may be recognized by the body as “injury” to the basement membrane of the vessel. Thus regulation of MP activity in indications such as dilated cardiomyopathy, congestive heart failure, atherosclerosis, plaque rupture, reperfusion injury, ischemia, chronic obstructive pulmonary disease, angioplasty restenosis and aortic aneurysm may increase long term success of any other treatment, or may be a treatment in itself. MMPs are implicated in artherosclerotic plaque rupture. See e.g., Galis, Z. S., et al., J. Clin. Invest. 1994; 94:2493-503; Lee, R. T., et al., Arterioscler. Thromb. Vasc. Biol. 1996; 16:1070-73; Schonbeck, U. et al., Circulation Research 1997; 81(3), 448-454. Libby, P. et al., Circ. 1995; 91:2844-50.

In skin care, MPs are implicated in the remodeling or “turnover” of skin. As a result, the regulation of MPs improves treatment of skin conditions including, but not limited to, wrinkle repair, regulation and prevention and repair of ultraviolet induced skin damage. Such a treatment includes prophylactic treatment or treatment before the physiological manifestations are obvious. For example, the MP may be applied as a pre-exposure treatment to prevent ultraviolet damage and/or during or after exposure to prevent or minimize post-exposure damage. In addition, MPs are implicated in skin disorders and diseases related to abnormal tissues that result from abnormal turnover, which includes metalloprotease activity, such as epidermolysis bullosa, psoriasis, scleroderma and atopic dermatitis. The compounds of the invention are also useful for treating the consequences of “normal” injury to the skin including scarring or “contraction” of tissue, for example, following burns. MP inhibition is also useful in surgical procedures involving the skin for prevention of scarring, and promotion of normal tissue growth including in such applications as limb reattachment and refractory surgery (whether by laser or incision).

In addition, MPs are related to disorders involving irregular remodeling of other tissues, such as bone, for example, in osteosclerosis and/or osteoporosis, or for specific organs, such as in liver cirrhosis and fibrotic lung disease. Similarly, in diseases such as multiple sclerosis, MPs may be involved in the irregular modeling of blood brain barrier and/or myelin sheaths of nervous tissue. Thus, regulating MP activity may be used as a strategy in treating, preventing, and controlling such diseases.

MPs are also thought to be involved in many infections, including cytomegalovirus (CMV); retinitis; HIV, and the resulting syndrome, AIDS.

MPs may also be involved in extra vascularization where surrounding tissue needs to be broken down to allow new blood vessels such as in angiofibroma and hemangioma.

Since MPs break down the extracellular matrix, it is contemplated that inhibitors of these enzymes can be used as birth control agents, for example in preventing ovulation, in preventing penetration of the sperm into and through the extracellular milieu of the ovum, implantation of the fertilized ovum and in preventing sperm maturation. Additionally, they are also contemplated to be useful in preventing or stopping premature labor and delivery.

Since MPs are implicated in the inflammatory response and in the processing of cytokines, the compounds are also useful as anti-inflammatories, for use in disease where inflammation is prevalent including, inflammatory bowel disease, Crohn's disease, ulcerative colitis, pancreatitis, diverticulitis, asthma or related lung disease, rheumatoid arthritis, gout and Reiter's Syndrome.

Where autoimmunity is the cause of the disorder, the immune response often triggers MP and cytokine activity. Regulation of MPs in treating such autoimmune disorders is a useful treatment strategy. Thus MP inhibitors can be used for treating disorders including, lupus erythematosus, ankylosing spondylitis, and autoimmune keratitis. Sometimes the side effects of autoimmune therapy result in exacerbation of other conditions mediated by MPs, here MP inhibitor therapy is effective as well, for example, in autoimmune-therapy-induced fibrosis.

In addition, other fibrotic diseases lend themselves to this type of therapy, including pulmonary disease, bronchitis, emphysema, cystic fibrosis and acute respiratory distress syndrome (especially the acute phase response).

Where MPs are implicated in the undesired breakdown of tissue by exogenous agents, these can be treated with MP inhibitors. For example, they are effective as rattle snake bite antidote, as anti-vessicants, in treating allergic inflammation, septicemia and shock. In addition, they are useful as antiparasitics (e.g., in malaria) and antiinfectives. For example, they are thought to be useful in treating or preventing viral infection, including infection which would result in herpes, “cold” (e.g., rhinoviral infection), meningitis, hepatitis, HIV infection and AIDS.

MP inhibitors are also thought to be useful in treating Alzheimer's disease, amyotrophic lateral sclerosis (ALS), muscular dystrophy, complications resulting from or arising out of diabetes, especially those involving loss of tissue viability, coagulation, Graft vs. Host disease, leukemia, cachexia, anorexia, proteinuria, and regulation of hair growth.

For some diseases, conditions or disorders MP inhibition is contemplated to be a preferred method of treatment. Such diseases, conditions or disorders include, arthritis (including osteoarthritis and rheumatoid arthritis), cancer (especially the prevention or arrest of tumor growth and metastasis), ocular disorders (especially corneal ulceration, lack of corneal healing, macular degeneration, and pterygium), and gum disease (especially periodontal disease, and gingivitis)

Compounds preferred for, but not limited to, the treatment of arthritis (including osteoarthritis and rheumatoid arthritis) are those compounds that are selective for the matrix metalloproteases and the disintegrin metalloproteases. Compounds preferred for, but not limited to, the treatment of cancer (especially the prevention or arrest of tumor growth and metastasis) are those compounds that preferentially inhibit gelatinases or type IV collagenases. Compounds preferred for, but not limited to, the treatment of ocular disorders (especially corneal ulceration, lack of corneal healing, macular degeneration, and pterygium) are those compounds that broadly inhibit metalloproteases. Preferably these compounds are administered topically, more preferably as a drop or gel. Compounds preferred for, but not limited to, the treatment of gum disease (especially periodontal disease, and gingivitis) are those compounds that preferentially inhibit collagenases.

One of ordinary skill in the art will also appreciate that when using the compounds of the invention in the treatment of a specific disease that the compounds of the invention may be combined with various existing therapeutic agents used for that disease. For the treatment of rheumatoid arthritis, the compounds of the invention may be combined with agents such as TNF-a inhibitors such as anti-TNF monoclonal antibodies and TNF receptor immunoglobulin molecules (such as ENBREL™), low dose methotrexate, lefunimide, hydroxychloroquine, d-penicillamine, auranofin or parenteral or oral gold.

The compounds of the invention can also be used in combination with existing therapeutic agents for the treatment of osteoarthritis. Suitable agents to be used in combination include standard non-steroidal anti-inflammatory agents (hereinafter NSAID's) such as piroxicam, diclofenac, propionic acids such as naproxen, flurbiprofen, fenoprofen, ketoprofen and ibuprofen, fenamates such as mefenamic acid, indomethacin, sulindac, apazone, pyrazolones such as phenylbutazone, salicylates such as aspirin, COX-2 inhibitors such as etoricoxib and rofecoxib, analgesics and intraarticular therapies such as corticosteroids and hyaluronic acids such as hyalgan and synvisc.

This invention also relates to a method of or a pharmaceutical composition for treating inflammatory processes and diseases comprising administering a compound of this invention to a mammal, including a human, cat, livestock or dog, wherein said inflammatory processes and diseases are defined as above and said inhibitory compound is used in combination with one or more other therapeutically active agents under the following conditions: A.) where a joint has become seriously inflamed as well as infected at the same time by bacteria, fungi, protozoa and/or virus, said inhibitory compound is administered in combination with one or more antibiotic, antifungal, antiprotozoal and/or antiviral therapeutic agents; B) where a multi-fold treatment of pain and inflammation is desired, said inhibitory compound is administered in combination with inhibitors of other mediators of inflammation; where older mammals are being treated for disease conditions, syndromes and symptoms found in geriatric mammals, for example, cognitive therapeutics to counteract memory loss and impairment; anti-hypertensives and other cardiovascular drugs intended to offset the consequences of atherosclerosis, hypertension, myocardial ischemia, angina, congestive heart failure and myocardial infarction,

The active ingredient of the present invention may be administered in combination with inhibitors of other mediators of inflammation, comprising one or more members selected from the group consisting essentially of the classes of such inhibitors and examples thereof which include, matrix metalloproteinase inhibitors, aggrecanase inhibitors, TACE inhibitors, leucotriene receptor antagonists, IL-1 processing and release inhibitors, Lira, H₁-receptor antagonists; kinin-B₁- and B₂-receptor antagonists; prostaglandin inhibitors such as PGD-, PGF-PGI₂- and PGE-receptor antagonists; thromboxane A₂ (TXA2-) inhibitors; 5- and 12-lipoxygenase inhibitors; leukotriene LTC₄-, LTD4/LTE₄- and LTB₄-inhibitors; PAF-receptor antagonists; gold in the form of an aurothio group together with various hydrophilic groups; immunosuppressive agents, e.g., cyclosporine, azathioprine and methotrexate; anti-inflammatory glucocorticoids; penicillamine; hydroxychloroquine; anti-gout agents, e.g., colchicine, xanthine oxidase inhibitors, e.g., allopurinol and uricosuric agents, e.g., probenecid, sulfinpyrazone and benzbromarone.

The compounds of the present invention may also be used in combination with anticancer agents such as endostatin and angiostatin or cytotoxic drugs such as adriamycin, daunomycin, cis-platinum, etoposide, taxol, taxotere and alkaloids, such as vincristine and antimetabolites such as methotrexate.

The compounds of the present invention may also be used in combination with anti-hypertensives and other cardiovascular drugs intended to offset the consequences of atherosclerosis, including hypertension, myocardial ischemia including angina, congestive heart failure and myocardial infarction, selected from vasodilators such as hydralazine, β-adrenergic receptor antagonists such as propranolol, calcium channel blockers such as nifedipine, α₂-adrenergic agonists such as clonidine, α-adrenergic receptor antagonists such as prazosin and HMG-CoA-reductase inhibitors (anti-hypercholesterolemics) such as lovastatin or atorvastatin.

The compounds of the present invention may also be administered in combination with one or more antibiotic, antifungal, antiprotozoal, antiviral or similar therapeutic agents.

The compounds of the present invention may also be used in combination with CNS agents such as antidepressants (such as sertraline), anti-Parkinsonian drugs (such as L-dopa, requip, mirapex, MAOB inhibitors such as selegine and rasagiline, comP inhibitors such as Tasmar, A-2 inhibitors, dopamine reuptake inhibitors, NMDA antagonists, nicotine agonists, dopamine agonists and inhibitors of neuronal nitric oxide synthase) and anti-Alzheimer's drugs such as donepezil, tacrine, COX-2 inhibitors, propentofylline or metrifonate.

The compounds of the present invention may also be used in combination with osteoporosis agents such as roloxifene, lasofoxifene, droloxifene or fosomax and immunosuppressant agents such as FK-506 and rapamycin.

The invention compounds may be used in combination with a COX-2 selective inhibitor, more preferably celecoxib (e.g., CELEBREX™), valdecoxib (e.g., BEXTRA™), parecoxib, lumiracoxib (e.g., PREXIGE™), or rofecoxib (e.g., VIOXX™), or with compounds such as etanercept (e.g., ENBREL™, infliximab (e.g., REMICADE™), leflunomide, (e.g., ARAVA™) or methotrexate, and the like.

The invention compounds may be used in combination with biological therapeutics useful for treating arthritic conditions, including CP-870, etanercept (a tumor necrosis factor alpha (“TNF-alpha”) receptor immunoglobulin molecule; trade names ENBREL™ and ENBREL ENTANERCEPT™ by Immunex Corporation, Seattle, Wash.), infliximab (an anti-TNF-alpha chimeric IgG 1K monoclonal antibody; tradename REMICADE™ by Centocor, Inc., Malvern, Pa.), methotrexate (tradename RHEUMATREX™ by American Cyanamid Company, Wayne, N.J.), and adalimumab (a human monoclonal anti-TNF-alpha antibody; tradename HUMIRA™ by Abbott Laboratories, Abbott Park, Ill.).

The present invention also relates to the formulation of a compound of the present invention alone or with one or more other therapeutic agents which are to form the intended combination, including wherein said different drugs have varying half-lives, by creating controlled-release forms of said drugs with different release times which achieves relatively uniform dosing; or, in the case of non-human patients, a medicated feed dosage form in which said drugs used in the combination are present together in admixture in the feed composition. There is further provided in accordance with the present invention co-administration in which the combination of drugs is achieved by the simultaneous administration of said drugs to be given in combination; including co-administration by means of different dosage forms and routes of administration; the use of combinations in accordance with different but regular and continuous dosing schedules whereby desired plasma levels of said drugs involved are maintained in the patient being treated, even though the individual drugs making up said combination are not being administered to said patient simultaneously.

The invention method is useful in human and veterinary medicines for treating mammals suffering from one or more of the above-listed diseases and disorders.

All that is required to practice a method of this invention is to administer a compound of the invention or a pharmaceutically acceptable composition thereof, in an amount that is therapeutically effective for preventing, inhibiting, or reversing the condition being treated. The invention compound can be administered directly or in a pharmaceutical composition as described below.

A therapeutically effective amount, or, simply, effective amount, of an invention compound will generally be from about 1 to about 300 mg/kg of subject body weight of the compound of the invention, or a pharmaceutically acceptable salt thereof. Typical doses will be from about 10 to about 5000 mg/day for an adult subject of normal weight for each component of the combination. In a clinical setting, regulatory agencies such as, for example, the Food and Drug Administration (“FDA”) in the U.S. may require a particular therapeutically effective amount.

A “safe and effective amount” of a compound of the invention, is an amount that is effective to inhibit metalloproteases at the site(s) of activity in an animal, preferably a mammal, more preferably a human subject, without undue adverse side effects (such as toxicity, irritation, or allergic response), commensurate with a reasonable benefit/risk ratio when used in the manner of this invention. The specific “safe and effective amount” will, obviously, vary with such factors as the particular condition being treated, the physical condition of the patient, the duration of treatment, the nature of concurrent therapy (if any), the specific dosage form to be used, the carrier employed, the solubility of the compound therein, and the dosage regimen desired for the composition.

In determining what constitutes a nontoxic effective amount or a therapeutically effective amount of an invention compound for treating, preventing, or reversing one or more symptoms of any one of the diseases and disorders described above that are being treated according to the invention methods, a number of factors will generally be considered by the medical practitioner or veterinarian in view of the experience of the medical practitioner or veterinarian, including the Food and Drug Administration guidelines, or guidelines from an equivalent agency, published clinical studies, the subject's (e.g., mammal's) age, sex, weight and general condition, as well as the type and extent of the disease, disorder or condition being treated, and the use of other medications, if any, by the subject. As such, the administered dose may fall within the ranges or concentrations recited above, or may vary outside them, i.e., either below or above those ranges, depending upon the requirements of the individual subject, the severity of the condition being treated, and the particular therapeutic formulation being employed. Determination of a proper dose for a particular situation is within the skill of the medical or veterinary arts. Generally, treatment may be initiated using smaller dosages of the invention compound that are less than optimum for a particular subject. Thereafter, the dosage can be increased by small increments until the optimum effect under the circumstance is reached. For convenience, the total daily dosage may be divided and administered in portions during the day, if desired.

Pharmaceutical compositions, described briefly here and more fully below, of an invention combination may be produced by formulating the invention combination in dosage unit form with a pharmaceutical carrier. Some examples of dosage unit forms are tablets, capsules, pills, powders, aqueous and nonaqueous oral solutions and suspensions, and parenteral solutions packaged in containers containing either one or some larger number of dosage units and capable of being subdivided into individual doses. Alternatively, the invention compounds may be formulated separately.

Some examples of suitable pharmaceutical carriers, including pharmaceutical diluents, are gelatin capsules; sugars such as lactose and sucrose; starches such as corn starch and potato starch; cellulose derivatives such as sodium carboxymethyl cellulose, ethyl cellulose, methyl cellulose, and cellulose acetate phthalate; gelatin; talc; stearic acid; magnesium stearate; vegetable oils such as peanut oil, cottonseed oil, sesame oil, olive oil, corn oil, and oil of theobroma; propylene glycol, glycerin; sorbitol; polyethylene glycol; water; agar; alginic acid; isotonic saline, and phosphate buffer solutions; as well as other compatible substances normally used in pharmaceutical formulations.

The compositions to be employed in the invention can also contain other components such as coloring agents, flavoring agents, and/or preservatives. These materials, if present, are usually used in relatively small amounts. The compositions can, if desired, also contain other therapeutic agents commonly employed to treat any of the above-listed diseases and disorders.

Compositions of this invention can be administered topically or systemically. Systemic application includes any method of introducing the inventive compounds into the tissues of the body, e.g., intra-articular (especially in treatment of rheumatoid arthritis), intrathecal, epidural, intramuscular, transdermal, intravenous, intraperitoneal, subcutaneous, sublingual, rectal, and oral administration. Preferred routes of administration of an invention compound are oral or parenteral. However, another route of administration may be preferred depending upon the condition being treated. For example, topical administration or administration by injection may be preferred for treating conditions localized to the skin or a joint. Administration by transdermal patch may be preferred where, for example, it is desirable to effect sustained dosing.

It should be appreciated that the different routes of administration may require different dosages. For example, a useful intravenous (“IV”) dose is between 5 and 50 mg, and a useful oral dosage is between 20 and 800 mg, of a compound of the invention, e.g. SEQ ID NOS: 1-6, or a pharmaceutically acceptable salt thereof. The dosage is within the dosing range used in treatment of the above-listed diseases, or as would be determined by the needs of the patient as described by the physician.

The invention compounds may be administered in any form. Preferably, administration is in unit dosage form. A unit dosage form of the invention compound to be used in this invention may also comprise other compounds useful in the therapy of diseases described above. A further description of pharmaceutical formulations useful for administering the invention compounds and invention combinations is provided below.

The active components of the invention combinations, may be formulated together or separately and may be administered together or separately. The particular formulation and administration regimens used may be tailored to the particular patient and condition being treated by a practitioner of ordinary skill in the medical or pharmaceutical arts. The advantages of using an invention compound in a method of the instant invention include the nontoxic nature of the compounds at and substantially above therapeutically effective doses, their ease of preparation, the fact that the compounds are well-tolerated, and the ease of topical, IV, or oral administration of the drugs.

Preparations of the invention compounds may use starting materials, reagents, solvents, and catalysts that may be purchased from commercial sources or they may be readily prepared by adapting procedures in the references or resources cited above. Commercial sources of starting materials, reagents, solvents, and catalysts useful in preparing invention compounds include, for example, The Aldrich Chemical Company, and other subsidiaries of Sigma-Aldrich Corporation, St. Louis, Mo., BACHEM, BACHEM A. G., Switzerland, or Lancaster Synthesis Ltd, United Kingdom.

Syntheses of some invention compounds may utilize starting materials, intermediates, or reaction products that contain a reactive functional group. During chemical reactions, a reactive functional group may be protected from reacting by a protecting group that renders the reactive functional group substantially inert to the reaction conditions employed. A protecting group is introduced onto a starting material prior to carrying out the reaction step for which a protecting group is needed. Once the protecting group is no longer needed, the protecting group can be removed. It is well within the ordinary skill in the art to introduce protecting groups during a synthesis of a compound of the invention, or a pharmaceutically acceptable salt thereof, and then later remove them. Procedures for introducing and removing protecting groups are known and referenced such as, for example, in Protective Groups in Organic Synthesis, 2nd ed., Greene T. W. and Wuts P. G., John Wiley & Sons, New York: N.Y., 1991, which is hereby incorporated by reference.

Thus, for example, protecting groups such as the following may be utilized to protect amino, hydroxyl, and other groups: carboxylic acyl groups such as, for example, formyl, acetyl, and trifluoroacetyl; alkoxycarbonyl groups such as, for example, ethoxycarbonyl, tert-butoxycarbonyl (BOC), .βββ-trichloroethoxycarbonyl (TCEC), and β-iodoethoxycarbonyl; aralkyloxycarbonyl groups such as, for example, benzyloxycarbonyl (CBZ), para-methoxybenzyloxycarbonyl, and 9-fluorenylmethyloxycarbonyl (FMOC); trialkylsilyl groups such as, for example, trimethylsilyl (TMS) and tert-butyldimethylsilyl (TBDMS); and other groups such as, for example, triphenylmethyl (trityl), tetrahydropyranyl, vinyloxycarbonyl, ortho-nitrophenylsulfenyl, diphenylphosphinyl, para-toluenesulfonyl (Ts), mesyl, trifluoromethanesulfonyl, and benzyl. Examples of procedures for removal of protecting groups include hydrogenolysis of CBZ groups using, for example, hydrogen gas at 50 psi in the presence of a hydrogenation catalyst such as 10% palladium on carbon, acidolysis of BOC groups using, for example, hydrogen chloride in dichloromethane, trifluoroacetic acid (TFA) in dichloromethane, and the like, reaction of silyl groups with fluoride ions, and reductive cleavage of TCEC groups with zinc metal.

The following examples are offered by way of illustration, not by way of limitation. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification.

All publications and patent documents cited in this application are incorporated by reference in pertinent part for all purposes to the same extent as if each individual publication or patent document were so individually denoted. By their citation of various references in this document, Applicants do not admit any particular reference is “prior art” to their invention.

### EXAMPLES

### Materials and Methods

All standard chemicals were peptide synthesis or molecular biology grade and purchased from Fisher Scientific. 2-(1H-benzotriazole-1-yl)-1,1,3,3-tetramethyluronium hexafluorophosphate, 1-hydroxybenzotriazole, and Fmoc-amino acid derivatives were obtained from Novabiochem (San-Diego, Calif.). Amino acids are of L-configuration (except for Gly). Mca-Lys-Pro-Leu-Gly-Leu-Lys(Dnp)-Ala-Arg-NH₂ was synthesized by methods described previously.

Peptide Synthesis: Peptide-resin assembly of triple-helical peptides (THPs) was performed by Fmoc solid-phase methodology on an Applied Biosystems Pioneer Peptide Synthesizer. All peptides were synthesized as C-terminal amides using Fmoc-PAL-PEG-PS resin to prevent diketopiperazine formation. Peptide-resins were lipidated with hexanoic acid [CH₃(CH₂)₄CO₂H, designated C₆]. Cleavage and side-chain deprotection of peptide-resins proceeded for at least 3 h using thioanisole-water-TFA (5:5:90). Cleavage solutions were extracted with methyl tBu ether prior to purification.

Peptide Purification: RP-HPLC purification was performed on a Rainin AutoPrep System with a Vydac 218TP152022 C₁₈ column (15-20 μm particle size, 300 Å pore size, 250×22 mm) at a flow rate of 10.0 mL/min. Eluants were 0.1% TFA in water (A) and 0.1% TFA in acetonitrile (B). The elution gradient was adjusted as required. Detection was at λ=220 nm. Analytical RP-HPLC and MALDI-TOF MS (see below) were used to identify fractions of homogenous product.

Peptide Analyses: Analytical RP-HPLC was performed on a Hewlett-Packard 1100 Liquid Chromatograph equipped with a Vydac 218TP5415 C₁₈ RP column (5 μm particle size, 300 Å pore size, 150×4.6 mm). Eluants were 0.1% TFA in water (A) and 0.1% TFA in acetonitrile (B). The elution gradient was 0-100% B in 20 min with a flow rate of 1.0 mL/min. Detection was at λ=220, 324, and 363 nm. MALDI-TOF-MS was performed on a Applied Biosystems Voyager MALDI-TOF mass spectrometer using α-cyano-4-hydroxycinnamic acid matrix. Mass values were as follows: f1, [M+H]⁺ 3579.2 Da (theoretical 3577.9 Da); f2, [M+H]⁺ 3579.2 Da (theoretical 3577.9 Da); f3, [M+H]⁺ 3673.4 Da (theoretical 3676.1 Da); and f4, [M+H]⁺ 3673.4 Da (theoretical 3676.1 Da).

Circular Dichroism Spectroscopy: CD spectra were recorded over the range λ=190-250 nm on a JASCO J-810 spectropolarimeter using a 1.0 cm path-length quartz cell. Thermal transition curves were obtained by recording the molar ellipticity ([Θ]) at λ=222 nm while the temperature was continuously increased in the range of 5-95° C. at a rate of 0.2° C./min. Temperature was controlled using a JASCO PFD-425S temperature control unit. For samples exhibiting sigmoidal melting curves, the inflection point in the transition region (first derivative) is defined as the melting temperature (Tₘ).

Matrix Metalloproteinases: ProMMP-1 and proMMP-3 were expressed in E. coli and folded from the inclusion bodies as described previously. ProMMP-1 was activated by reacting with 1 mM APMA and 0.1 equiv of MMP-3(Δ₂₄₈₋₄₆₀) at 37° C. for 6 h. After activation, MMP-3(Δ₂₄₈₋₄₆₀) was completely removed from MMP-1 by affinity chromatography using an anti-MMP-3 IgG Affi-Gel 10 column. ProMMP-3 was activated by reacting with 5 μg/mL chymotrypsin at 37° C. for 2 h. Chymotrypsin was inactivated with 2 mM diisopropylfluorophosphate. ProMMP-2 was purified from the culture medium of human uterine cervical fibroblasts and activated by incubating with 1 mM APMA for 2 h at 37° C. ProMMP-8 was expressed in CHO-K¹ cells as described previously. ProMMP-8 was activated by incubating with 1 mM APMA for 2 h at 37° C. Recombinant proMMP-9 was purchased from Chemicon International (Temecula, Calif.) and activated with 1 mM APMA at 37° C. ProMMP-13 was purchased from R&D Systems (Minneapolis, Minn.) and activated by incubating with 1 mM APMA for 2 h at 37° C. The concentrations of active MMP-1, MMP-2, MMP-3, MMP-8, MMP-9(Δ₄₄₄₋₇₀₇), and MMP-13 were determined by titration with recombinant TIMP-1 or N-TIMP-1 over a concentration range of 0.1-3 μg/mL. Recombinant MT1-MMP with the linker and C-terminal hemopexin-like domains deleted [residues 279-523; designated MT1-MMP(A₂₇₉₋₅₂₃)] was purchased from Chemicon. MT1-MMP(Δ₂₇₉₋₅₂₃) was expressed and activated, resulting in Tyr112 at the N-terminus. MT1-MMP(Δ₂₇₉₋₅₂₃), which, in contrast to MT1-MMP, does not undergo rapid autoproteolysis, was used in the present studies due to the relatively small differences in MT1-MMP(Δ₂₇₉₋₅₂₃) and MT1-MMP triple-helical peptidase activities noted previously. The concentration of active MT1-MMP(Δ₂₇₉₋₅₂₃) was determined by titration with recombinant TIMP-2, N-TIMP-2, or N-TIMP-3. ProMMP-3(Δ₂₄₈₋₄₆₀) were expressed in E. coli using the expression vector pET3a (Novagen), folded from inclusion bodies and purified as described previously. The zymogen was activated as described above for the full-length proMMP-3. Active site titrations utilized either Mca-Lys-Pro-Leu-Gly-Leu-Lys(Dnp)-Ala-Arg-NH₂ or NFF-3 as substrate.

Inhibition Kinetic Studies: Peptide substrates and inhibitors were dissolved in TNC buffer (50 mM tris-HCl, pH 7.5 containing 100 mM NaCl, 10 mM CaCl₂, 0.05% Brij-35, pH 7.5). 1-2 nM enzyme was incubated with varying concentrations of inhibitors for 2 h at room temperature. Residual enzyme activity was monitored by adding 0.1 volume of Mca-Lys-Pro-Leu-Gly-Leu-Lys(Dnp)-Ala-Arg-NH₂ to produce a final concentration of <0.1 K_{M}. Initial velocity rates were determined from the first 20 min of hydrolysis when product release is linear with time. Fluorescence was measured on a Molecular Devices SPECTRAmax Gemini EM Dual-Scanning Microplate Spectrofluorimeter using λ_{excitation}=324 nm and λₑₘᵢₛₛᵢₒₙ=393 nm. Apparent Kᵢ values were calculated using SigmaPlot® by fitting data to the Morrison equation. In cases where weak inhibition occurred, apparent Kᵢ values were calculated using the equation: v=v₀/(1+I/Kᵢ) where v₀ is the activity in the absence of inhibitor and Kᵢ is the apparent inhibition constant. Because the substrate concentration is less than K_{M}/10, apparent Kᵢ values are insignificantly different from true Kᵢ values.

Synthesis of (R,S)-2-Isopropyl-3-((1-(N-(9-Fluorenylmethoxycarbonyl)amino)-methyl)-adamantyloxyphosphinyl)propanoic acid (1): The synthesis of the protected phosphinate mimic (1) is shown in FIG. 1. 1-(N-(9-Fmoc)amino)-methyl phosphinic acid (5) was prepared as follows. Briefly, the phosphinate analog of Gly, 1-aminomethylphosphinic acid (4), is prepared by the method of Baylis et al. (Baylis et al. (1984) J. Chem. Soc., Perkin Trans. 1, 2845-2853) from the imine cyclotrimer 3 hyphophosphorus acid, followed by hydrolysis to cleave the benzhydryl group. Next, 4 is treated with TMS-Cl, DIEA, and Fmoc-Cl to give 1-(N-(9-Fmoc)amino)-methyl phosphinic acid (5).

To a suspension of t-BuOK (8.5 g, 72.0 mmol) in THF (200 mL) was added ethyl acetoacetate (9.9 mL, 70.4 mmol) at 0° C. The resulting clear solution was stirred for 30 min, and then iodopropane (13.7 mL, 134 mmol) was added to the solution. The solution was stirred at 70° C. for 12 h. The reaction was quenched with water, and then a saturated aqueous sodium bicarbonate solution was added. The aqueous layer was extracted with diethyl ether (3×100 mL). The combined organic extracts were dried over MgSO₄, filtered, and concentrated to give a yellow oil. The crude product was purified by silica gel column chromatography (1:5 EtOAc/hexanes) to give 2-isopropyl-3-oxobutyric acid allyl ester (9.4 g) in 72% yield.

To a stirred solution of 2-isopropyl-3-oxobutyric acid allyl ester (4.61 g, 25.0 mmol) in THF (170 mL) was added LHMDS (27.5 mL, 27.5 mmol, and 1.0 M solution in THF) at −78° C. The solution was stirred for 30 min, and then paraformaldehyde (3.5 g, excess) was added as a solid in one portion. The resulting suspension was stirred at room temperature for 12 h and then filtered through Celite to remove the excess paraformaldehyde. The filtrate was concentrated, and the residue was purified by column chromatography (1:9 EtOAc/hexanes) to give 2-isopropylacrylic acid allyl ester (6) (2.7 g) in 69% yield.

To an ice cold suspension of 5 (532 mg, 1.68 mmol) in CH₂Cl₂ (8 mL), N,N-diisopropylethylamine (0.94 mL, 5.38 mmol) and chlorotrimethylsilane (0.68 mL, 5.38 mmol) were added under argon atmosphere. This solution was stirred for 3 h at room temperature. Then, the mixture was cooled to 0° C. and compound 6 (310 mg, 2.02 mmol) was added dropwise for 30 min. When the addition was over, the solution was stirred for 36 h at 40° C. Then, absolute ethanol (2 mL) was added dropwise and the mixture was stirred for 15 min. The solvent was evaporated. To the residue, H₂O was added and the resulting suspension was acidified with 1 M HCl to pH 1 and extracted with ethyl acetate (3×10 mL). The combined organic layers were dried over Na₂SO₄ and concentrated to dryness. The oily residue was purified by column chromatography (7:0.4:0.4 chloroform/methanol/acetic acid) to give 7 (423 mg) crude in 54% yield.

Compound 7 (360 mg, 0.77 mmol) and 1-adamantyl bromide (199 mg, 0.92 mmol) were dissolved in chloroform (10 mL), and the reaction mixture was refluxed. To this refluxing mixture, silver oxide (214 mg, 0.92 mmol) was added in five equal portions over 50 min. The reaction mixture was refluxed for two additional hours. Then the solvents were removed in vacuo and the residue was treated with diethylether. The silver bromide and the excess of silver oxide were removed by filtration through celite. The filtrate was concentrated to dryness and the residue was purified by column chromatography (98:2 chloroform/2-propanol) to give (R,S)-2-isopropyl-3-((1-(N-(9-fluorenylmethoxycarbonyl)amino)-methyl)-adamantyloxyphosphinyl) propanoic acid, allyl ester (451 mg) crude in 97% yield.

[CpRu(CH₃CN)₃]PF₆ (8.7 mg, 0.02 mmol), quinaldic acid (3.5 mg, 0.02 mmol) and ethanol (3 mL) were placed in a 10 mL two-neck round bottom flask under argon stream. After standing for 30 min at 25° C., the reddish brown solution was transferred into a 25 mL two-neck round bottom flask containing (R,S)-2-isopropyl-3-((1-(N-(9-fluorenylmethoxycarbonyl)amino)-methyl)-adamantyloxyphosphinyl) propanoic acid, allyl ester (1.0 g, 1.65 mmol) and ethanol (15 mL). The brown solution was stirred for 12 h at 70° C. The reaction mixture was concentrated under reduced pressure to give a crude product. This was purified by column chromatography (95:5 chloroform/2-propanol) to give 1 (332 mg) crude in 35% yield.

### Example 1

### Design of MMP Inhibitors

Initial clinical trials with MMP inhibitors showed that these inhibitors lacked selectivity. To date, the vast majority of MMP inhibitors contain a hydroxamic acid group which chelates the active site zinc. However, the hydroxamic acid usually represents a terminal point in the chain, and thus residues that interact with only one side of the enzyme active site can be incorporated. One way to circumvent this problem, and thus add sequence diversity, is to use an inhibitory molecule that, rather than terminate a chain, can be incorporated within a chain. In addition, inhibitors that recognize secondary substrate binding sites (exosites) can enhance selectivity. FIG. 1 is a schematic illustration showing the synthesis of phosphinate dipeptide mimic. FIG. 2 is a schematic illustration showing the preparation of the phosphonate dipeptide analog.

Two classes of proteases, the aspartyl proteases and the metallo(zinc)-proteases use an acid catalyzed addition of water as one of the steps of the amide bond hydrolysis. The tetrahedral intermediate that results from water addition to the amide carbonyl has been the focus of many protease inhibitor designs and has given rise to two robust classes of inhibitors, namely the statines and phosphorus-based amide bond replacements. An effective enzyme substrate almost invariably produces an inhibitor of the enzyme by incorporation of a statine (mainly for aspartyl proteases) or phosphorus (aspartyl and Zn²⁺ proteases) tetrahedral intermediate mimic. Thiophosphonates and thiophosphinates have been studied less, but hold great promise as inhibitors due to the increased affinity of the sulfur atom for zinc in the enzyme active site and the increased hydrophobicity of the thio-derivatives.

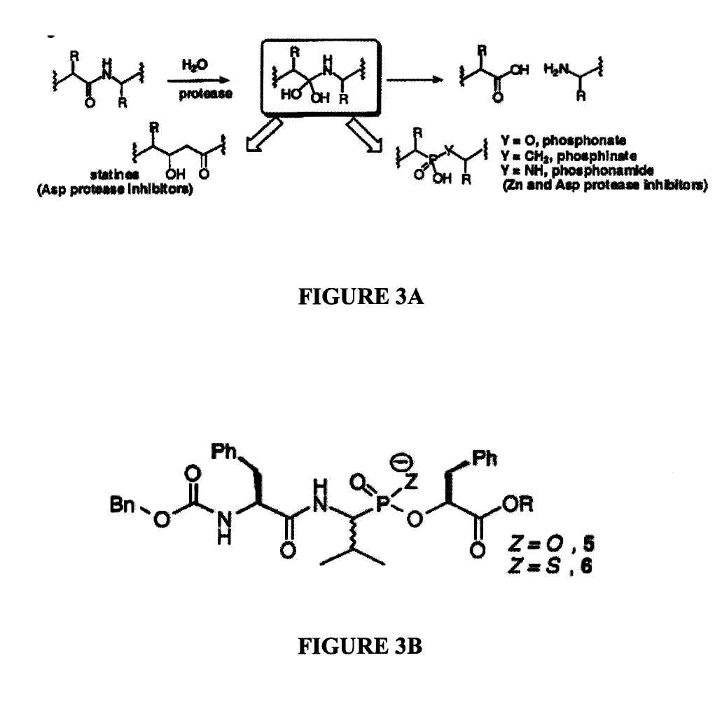

There are three general types of phosphorus-based inhibitors: phosphonamides [Ψ{PO₂H—NH}; Y═NH in FIG. 3A], phosphinic peptides [Ψ((PO₂H—CH₂); Y═CH₂ in FIG. 3A], and phosphonate esters [Ψ{PO₂H-Q}; Y=O in FIG. 3A]. Phosphonamidates have been shown previously to inhibit MMP-3 and MMP-8 by behaving as transition state analogs. Unfortunately, phosphonamides tend to be the least stable of the three phosphorus-based inhibitors. Phosphinic peptide combinatorial libraries have been used to design selective MMP-12 inhibitors with Kᵢ values in the low nM range. Phosphinic peptide inhibitors have also been described for MMP-2, MMP-8, MMP-9, MMP-11, and MMP-14. The most effective of these inhibitors had K values in the low nM range but, in general, were not particularly selective between the five aforementioned MMPs. A phosphonate peptide inhibitor has been described for MMP-8, (D'Alessio, S., et al. (1999), Bioorg. Med. Chem. Lett. 7, 389-394; Gavuzzo, E., et al. (2000) J. Med. Chem. 43, 3377-3385) but the selectivity was not investigated.

One reason that phosphorus-based inhibitor class has not been further exploited within the MMP family is the synthetic challenge of incorporating phosphonates within a peptide chain. Current methods that rely on coupling of heteroatom nucleophiles to P(V) derivatives are inefficient. A recently developed method starts with the P(lll) H-phosphinates using dichlorotriphenylphosphorane to activate the H-phosphinates non-oxidatively to phosphorochloridites, which react rapidly even with sterically hindered alcohols, amines, and thiols (Rushing, S. D., and Hammer, R. P. (2001) Synthesis of phosphonamide and thiophosphonamide dipeptides, J. Am. Chem. Soc. 123, 4861-4862). These P(lll) intermediates can be sulfurized to provide the first general route to thio- and dithio-phosphonate and phosphonamide peptides. (Rushing et al., supra). Both 5 (R=H) and 6 (R=H) inhibit carboxypeptidase with Kᵢ<10 pM. By combining phosphonate and thiophosphonate chemistry with triple-helical peptide technology, additional triple-helical transition state analog inhibitors can be designed and tested.

### Example 2

### Design and Synthesis of Triple-Helical Transition State Analog Inhibitors

To better mimic the natural substrate of MMPs, we have prepared phosphinate containing triple helical peptides. An Fmoc protected phosphino Gly-Val dipeptide mimic has been synthesized (FIG. 1) and incorporated into the triple helical collagen mimic sequence. Activity has also been examined herein as a function of triple-helical thermal stability. This has required the use of “peptide-amphiphiles,” in which the thermal stability of the triple-helix is modulated by pseudo-lipids attached to the N-terminus of the peptide. As both phosphinate and phosphonate mimics have been effective at inhibiting MMPs, both phosphinate and phosphonate triple-helical collagen substrate analogs described in Table 1 will be made. Additionally the thiophosphinate and thiophosphonate versions of peptide mimics which show promise as triple-helical inhibitors will be made. The MMP cleavage site in each of the sequences will be replaced by either a Gly-Leu phosphorus mimic or a Gly-Val phosphorus mimic [for α1(V)-436-450 sequence]. FIG. 4 shows the typical sequence that will be prepared using stepwise solid-phase peptide synthesis except that the Gly-Leu or Gly-Val phosphorus analog will be inserted as a phosphorus ester-protected, Fmoc-protected dipeptide 1 or 2 (FIG. 4).

TABLE 1
Phosphorus-based Tetrahedral Intermediate Analogs of Collagen Cleavage Sites^{a}

| | |
|---|---|
| Sequence Origin | Phosphinate or Phosphonate Analog of Cleaved Sequence |

| | | |
|---|---|---|
| α1(II)769-783 | Gly-Pro-Pro-Gly-Pro-Gln-Gly-ψ[P(O)(XH)-Y]Leu-Ala-Gly-Gln-Arg-Gly-Ile-Val | SEQ ID NO: 1 |
| α1(V)438-450 | Gly-Pro-Pro-Glyψ[P(O)(XH)-Y]Val-Val-Gly-Glu-Gln-Gly-Glu-Gln-Gly-Pro-Pro | SEQ ID NO: 2 |
| α1(IV)1366-1377 | Gly-Pro-Pro-Glyψ[P(O)(XH)-Y]Leu-Lys-Gly-Leu-Gln-Gly-Leu-Pro | SEQ ID NO: 3 |
| α1(IV)1426-1437 | Gly-Pro-Asp-Glyψ[P(O)(XH)-Y-]Leu-Pro-Gly-Ser-Met-Gly-Pro-Pro | SEQ ID NO: 4 |

When X = O: Y = CH₂, phosphinate: V = O, phoephonate. When X = S: Y = CH₂, thiophosphinate; Y = O, thiophosphonate.

The synthesis representative phosphinate mimics are shown in FIG. 1. The procedure follows the recently developed synthesis devised by Yiotakis and coworkers (Georgiadis, D., Matziari, M., and Yiotakis, A. (2001) Tetrahedron 57, 3471-3478). First, the phosphinate analog of glycine 4 is prepared by the method of Baylis et al. ((1984) 1-Aminoallylphosphonous acids, part 1: Isosteres of the protein amino acids, J. Chem. Soc., Perkin Trans. 1, 2845-2853) from the imine cyclotrimer 3 hyphophosphorus acid, followed by hydrolysis to cleave the benzhydryl group. Next, the phosphinate analog is protected as the Fmoc derivative, by treating 4 with TMS-Cl, DIEA, and Fmoc-Cl to give 1-(N-(9-Fmoc)amino)-methyl phosphinic acid (5).

To a suspension of t-BuOK (8.5 g, 72.0 mmol) in THF (200 mL) was added ethyl acetoacetate (9.9 mL, 70.4 mmol) at 0° C. The resulting clear solution was stirred for 30 min, and then iodopropane (13.7 mL, 134 mmol) was added to the solution. The solution was stirred at 70° C. for 12 h. The reaction was quenched with water, and then a saturated aqueous sodium bicarbonate solution was added. The aqueous layer was extracted with diethyl ether (3×100 mL). The combined organic extracts were dried over MgSO₄, filtered, and concentrated to give a yellow oil. The crude product was purified by silica gel column chromatography (1:5 EtOAc/hexanes) to give 2-isopropyl-3-oxobutyric acid allyl ester (9.4 g) in 72% yield.

To a stirred solution of 2-isopropyl-3-oxobutyric acid allyl ester (4.61 g, 25.0 mmol) in THF (170 mL) was added LHMDS (27.5 mL, 27.5 mmol, and 1.0 M solution in THF) at −78° C. The solution was stirred for 30 min, and then paraformaldehyde (3.5 g, excess) was added as a solid in one portion. The resulting suspension was stirred at room ternperature for 12 h and then filtered through Celite to remove the excess paraformaldehyde. The filtrate was concentrated, and the residue was purified by column chromatography (1:9 EtOAc/hexanes) to give 2-isopropylacrylic acid allyl ester (6) (2.7 g) in 69% yield.

To an ice cold suspension of 5 (532 mg, 1.68 mmol) in CH₂Cl₂ (8 mL), N,N-diisopropylethylamine (0.94 mL, 5.38 mmol) and chlorotrimethylsilane (0.68 mL, 5.38 mmol) were added under argon atmosphere. This solution was stirred for 3 h at room temperature. Then, the mixture was cooled to 0° C. and compound 6 (310 mg, 2.02 mmol) was added dropwise for 30 min. When the addition was over, the solution was stirred for 36 h at 40° C. Then, absolute ethanol (2 mL) was added dropwise and the mixture was stirred for 15 min. The solvent was evaporated. To the residue, H₂O was added and the resulting suspension was acidified with 1 M HCl to pH 1 and extracted with ethyl acetate (3×10 mL). The combined organic layers were dried over Na₂SO₄ and concentrated to dryness. The oily residue was purified by column chromatography (7:0.4:0.4 chloroform/methanol/acetic acid) to give 7 (423 mg) crude in 54% yield.

Mild silylation of the phosphinate with TMS-Cl and DIEA gives the trivalent phosphinate, which reacts readily in an Arbusov-like reaction with allyl acrylate 6 (R=iPr (Huntington, K. M., et al. (2000) Biochemistry 39, 4543-4551.) or R=iBu (Mori, K. et al. Tetrahedron 26, 2821-2824.)) to give the phosphinic acid 7. The acrylate esters are readily available from the nucleophilic esterification of the parent acids with allylbromide under phase-transfer conditions (Friedrich-Bochnitschek, S. et al. (1989) J. Org. Chem. 54, 751-756.). Finally, the phosphinate is protected as the adamantyl ester by reaction with silver oxide and adamantylbromide (Ad-Br) and the terminal allyl ester removed by Pd(0) catalysis to give the protected phosphinate dipeptide mimic 1. The thiophosphinate mimic is made by first activating the phosphinate to the acid chloride with thionyl chloride and then coupling with adamantanethiol, with final deblocking of the C-terminus by Pd(0) catalysis. The adamantyl ester does not prematurely hydrolyze but it is readily removed under TFA cleavage conditions used in Fmoc/tBu solid-phase strategies. (Yiotakis, A. et al. (1996) J. Org. Chem. 61, 6601-6605; Georgiadis, D. et al. (2001) J. Org. Chem. 66, 6604-6610). FIG. 4 shows a typical phosphinate triple-helical substrate analog to be prepared by solid-phase peptide synthesis. The method has provided overall yields of 70-80% from compound 5 to the desired product 1 for very similar substrates.

Compound 7 (360 mg, 0.77 mmol) and 1-adamantyl bromide (199 mg, 0.92 mmol) were dissolved in chloroform (10 mL), and the reaction mixture was refluxed. To this refluxing mixture, silver oxide (214 mg, 0.92 mmol) was added in five equal portions over 50 min. The reaction mixture was refluxed for two additional hours. Then the solvents were removed in vacuo and the residue was treated with diethylether. The silver bromide and the excess of silver oxide were removed by filtration through celite. The filtrate was concentrated to dryness and the residue was purified by column chromatography (98:2 chloroform/2-propanol) to give (R,S)-2-isopropyl-3-((1-(N-(9-fluorenylmethoxycarbonyl)amino)-methyl)-adamantyloxyphosphinyl) propanoic acid, allyl ester (451 mg) crude in 97% yield.

[CpRu(CH₃CN)₃]PF₆ (8.7 mg, 0.02 mmol), quinaldic acid (3.5 mg, 0.02 mmol) and ethanol (3 mL) were placed in a 10 mL two-neck round bottom flask under argon stream. After standing for 30 min at 25° C., the reddish brown solution was transferred into a 25 mL two-neck round bottom flask containing (R,S)-2-isopropyl-3-((1-(N-(9-fluorenylmethoxycarbonyl)amino)-methyl)-adamantyloxyphosphinyl) propanoic acid, allyl ester (1.0 g, 1.65 mmol) and ethanol (15 mL). The brown solution was stirred for 12 h at 70° C. The reaction mixture was concentrated under reduced pressure to give a crude product. This was purified by column chromatography (95:5 chloroform/2-propanol) to give 1 (332 mg) crude in 35% yield.

Synthesis and Characterization of Triple-Helical Transition State Analogs: N-Protected α-aminophosphinic acids are necessary intermediates in the synthesis of a variety of transition state mimics such as phoshinate, phosphonate, and phosphonamide dipeptide analogs and the 9-fluorenylmethoxycarbonyl (Fmoc) protecting group is ideal for peptide synthesis; however, there is no facile process currently reported for its incorporation onto such compounds. The previous methods using aqueous/organic solvent mixtures and classic were not generally high yielding due to hydrolysis side reactions and solubility problems. We hypothesized that use of anhydrous conditions would eliminate these difficulties. An in situ silylation procedure popularized by Bolin for L-amino acids (Bolin, 1989) was utilized for protection of α-aminophosphinic acid analog of Gly with an approximate yield of 80%. (Li, 2006).

Mild silylation of the phosphinate with TMS-Cl and DIEA gives the trivalent phosphinate, which reacts readily in an Arbusov-like reaction with allyl acrylate 6 (R=iPr to give the phosphinic acid 7. The acrylate esters are readily available from the nucleophilic esterification of the parent acids with allylbromide under phase-transfer conditions. Finally, the phosphinate is protected as the adamantyl ester by reaction with silver oxide and adamantylbromide (Ad-Br) and the terminal allyl ester removed by Pd(0) catalysis to give the protected phosphinate dipeptide mimic 1. The adamantyl ester does not prematurely hydrolyze but it is readily removed under TFA cleavage conditions used in Fmoc/tBu solid-phase strategies. The scheme shown has provided overall yields of 70-80% from compound 5 to the desired product 1 for very similar substrates.

It was previously shown that the type V collagen-derived sequence Gly-Pro-Pro-Gly₄₃₉-Val₄₄₀-Val-Gly-Glu-Gln, when incorporated into a triple-helical structure, was hydrolyzed efficiently by MMP-2 and MMP-9 but not by MMP-1, MMP-3, MMP-13, or MMP-14. The sequence was thus used as a template for the design of a potentially selective MMP transition state analog inhibitor. The Fmoc-protected phosphinate Gly-Val mimic 1 was prepared and incorporated by solid-phase methods to create (Gly-Pro-Hyp)-5-Gly-Pro-Pro-[Gly˜Val]-Val-Gly-Glu-Gln-(Gly-Pro-Hyp)₅-NH₂. The pseudo-peptide with Gly in P₁ and Val in P₁′ contains R and S isomers. A portion of the peptidyl-resin was lipidated on the N-terminus with hexanoic acid to create a peptide-amphiphile construct.

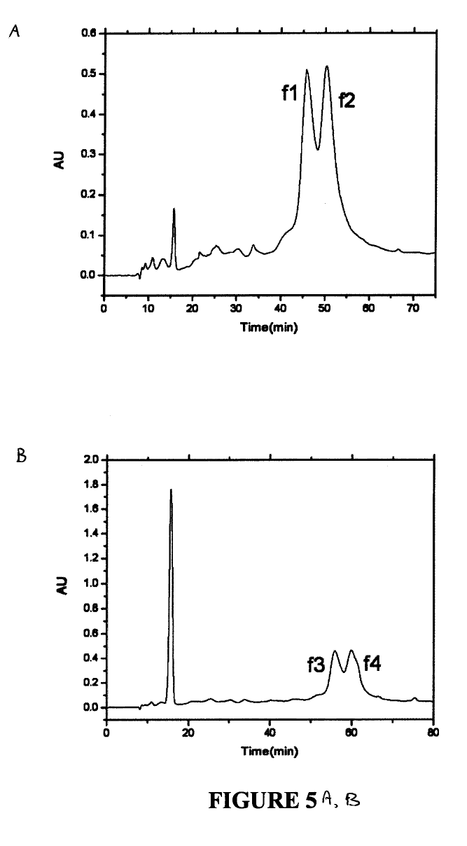

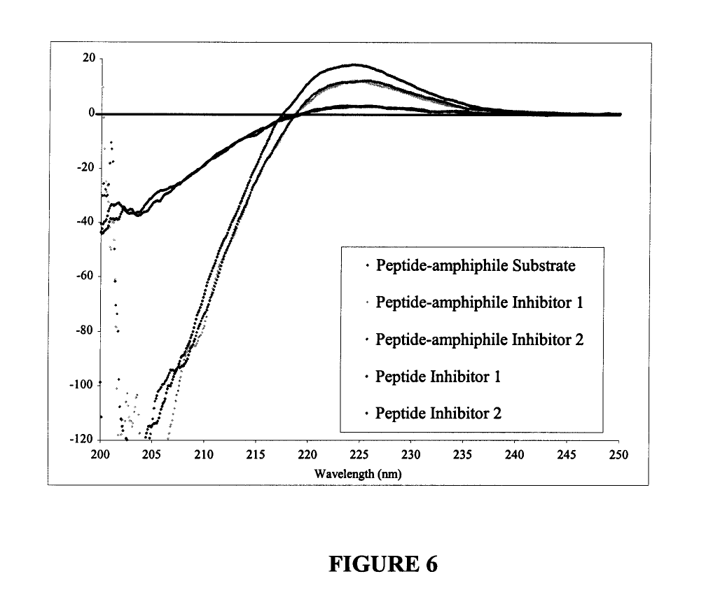

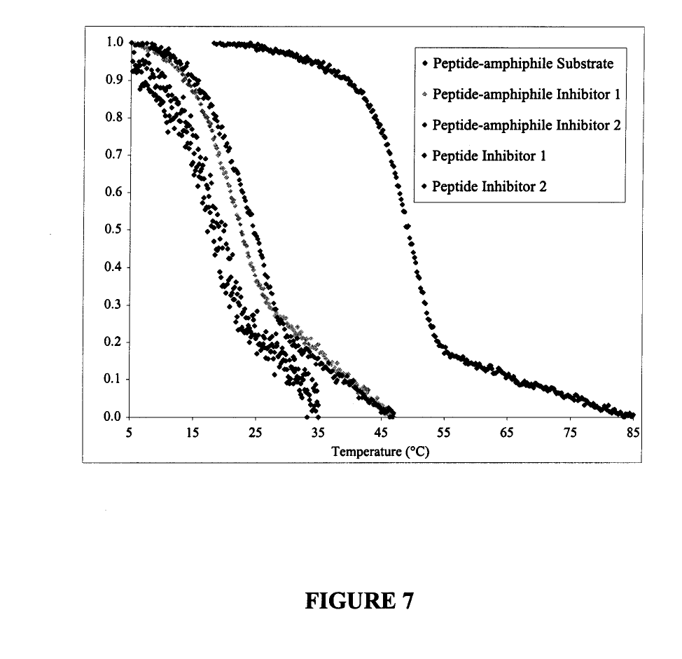

RP-HPLC analysis of the phosphinate-containing triple-helical peptide and peptide-amphiphile revealed two major peaks in each chromatogram (FIG. 5). The material in the major peaks (f1-f4) was isolated by RP-HPLC and analyzed by MALDI-TOF-MS and CD spectroscopy. Peaks f1 and f2 had masses that corresponded to the desired phosphinate-containing triple-helical peptide, while f3 and f4 had masses that corresponded to the desired phosphinate-containing triple-helical peptide-amphiphile. Thus, it appeared that in each case that the diastereomers could be separated. CD spectra indicated weak triple-helices for f1 and f2 and more pronounced triple-helical structure for f3 and f4 (FIG. 6). To examine the thermal stability of potential substrates, the molar ellipticity ([Θ]) at λ=225 nm was monitored as a function of increasing temperature. All structures exhibited cooperative transitions, indicative of the melting of a triple-helix to a single-stranded structure (FIG. 7). Melting temperatures (Tₘ values) were then determined for f1-f4. The f2 peptide had a slightly higher Tₘ than f1, while the f4 peptide-amphiphile had a higher Tₘ than f3. Based on this data, we hypothesize that f2 and f4 contain the phosphino Gly-(L)Val analog and that f1 and f3 contain the phosphino Gly-(D)Val analog.

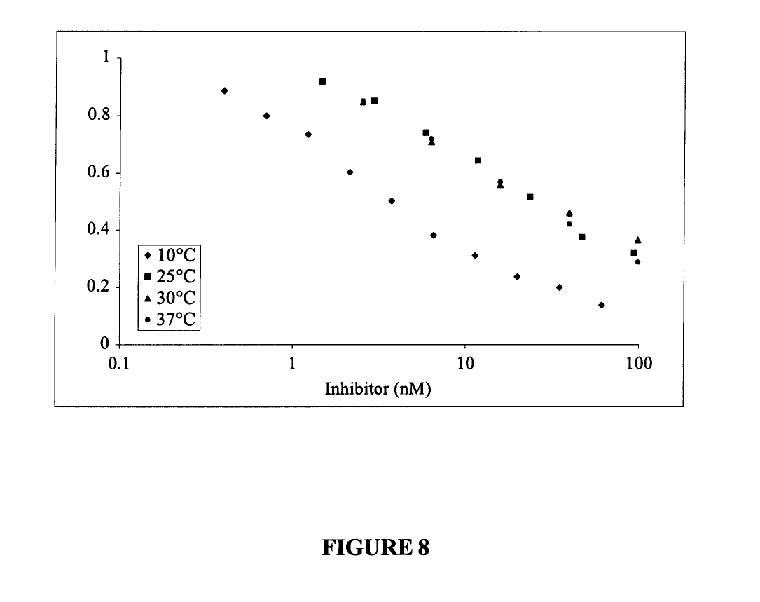

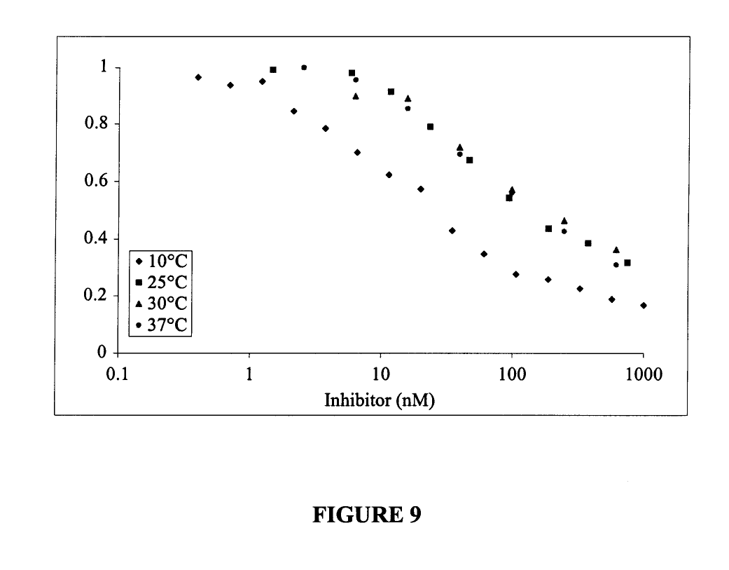

Inhibition of MMPs: Peptide-amphiphile mimics (f3 and f4) were initially tested against MMP-2 and MMP-9 (FIGS. 8 and 9). Due to the low melting temperatures of the potential inhibitors, Kᵢ values were first determined at 10° C. (Table 2). Both were found to be very effective inhibitors of MMP-2 and MMP-9, with Kᵢ values in the 4-6 and 2 nM ranges, respectively. When inhibition assays were repeated at 37° C., Kᵢ values increased for MMP-2 but not for MMP-9 (Table 2). Thus, triple-helical structure was important for inhibition of MMP-2, but not MMP-9. The increase in Kᵢ value as a function of temperature was not the same for f3 and f4. For f3, the increase in temperature resulted in a 5 times increase in Kᵢ for MMP-2 (Table 2). For f4, the increase in temperature resulted in a 25 times increase in Kᵢ for MMP-2 (Table 2).

To determine if an increase in Kᵢ as a function of temperature was general trend for inhibition of MMP-2, inhibition of MMP-2 by the MMP hydroxamic acid dipeptide inhibitor was examined. At 10° C., the Kᵢ value for MMP-2 inhibition was 3 nM (Table 2). Increasing the temperature to 37° C. decreased the Kᵢ to 0.8 mM (Table 2). Thus, for a small molecule inhibitor, an increase in temperature slightly increased the affinity towards MMP-2. Increased inhibition with increasing temperature is a general trend for enzymes. This further suggests that the decreased inhibition of MMP-2 by f3 and f4 as a function of increasing temperature is due to unfolding of the inhibitor triple-helical structure.

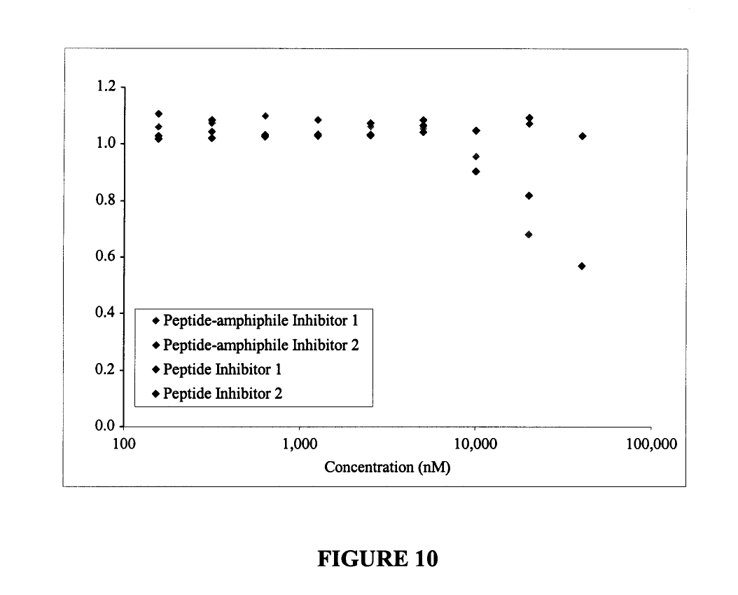

MMP-1, MMP-3, MMP-8, MMP-13, and MT1-MMP were tested for inhibition by f3 and f4. No inhibition of MMP-1, MMP-3, or MT1-MMP was observed. MMP-8 and MMP-13 were inhibited weakly, with IC₅₀ values in the range of 50 and 10 μM, respectively (FIG. 10).

Discussion: MMP-2 and -9 play an important role in metastasis. An MMP-2 and -9 selective substrate was used as a framework for a phosphinic acid-based inhibitor. Phosphinic acid pseudopeptides mimic the H₂O-bound tetrahedral transition state. An MMP-2 and MMP-9 selective inhibitor could be a (a) lead compound for drug development and (b) molecule allows us to study mechanism of collagenolysis as well as the importance of exosite interactions in inhibitor design.

Difference in stability between L- and D-form of the inhibitor is quite small. As shown previously, the use of stabilizing regions [such as (Gly-Pro-Hyp).] on both the N- and C-termini can fold and ordered a central non-Gly-Xxx-Yyy region, minimizing relative thermal destabilization.

These peptides have low nM Kᵢ values for MMP-2 and MMP-9 while have little or no activity against collagenolytic MMPs (MMP-1, MMP-8, MMP-13, and MT1-MMP). Triple-helical peptide inhibitors of MMPs have been constructed previously using a hydroxamate as the zinc binding group (ZBG). In one study, (Pro-Pro-Gly)₆-NHOH and (Pro-Pro-Gly)₁₂-NHOH were found to inhibit MMP-2 with IC₅₀ values of 160 and 90 μM, respectively. These constructs showed similarly weak inhibition of MMP-1 and MMP-3. The weak inhibition may have been the result of interaction with only the S subsites of the enzyme and/or poor alignment of the ZBG into the active site. A second study utilized a solid-phase C-terminal branching protocol to incorporate a hydroxamate-containing peptidomimetic onto the N-terminus of (Gly-Pro-Hyp)-4-Gly-Pro-Pro-Gly-Ser-Ser. Inhibition of MMP-1 was achieved with an IC₅₀ value of ˜9 nM. Constructs in which fewer Gly-Pro-Hyp repeats were present, and thus presumably had less or no triple-helicity, exhibited IC₅₀ values of ˜100-500 nM. Thus, MMP-1 inhibition was dependent upon triple-helical structure. While interesting, this particular inhibitor would offer little selectivity amongst collagenolytic MMPs.

TABLE 2
Inhibition of MMP-2 and MMP-2.

| | | Temperature | K_{i, app} |
|---|---|---|---|
| Enzyme | Inhibitor | (° C.) | (nM) |
| MMP-2 | Peptide-amphiphile 1 | 10 | 4.14 ± 0.47 |
| MMP-2 | Peptide-amphiphile 1 | 37 | 19.23 ± 0.64 |
| MMP-2 | Peptide-amphiphile 2 | 10 | 5.48 ± 0.00 |
| MMP-2 | Peptide-amphiphile 2 | 37 | 138.32 ± 27.85 |
| MMP-2 | Hydroxamic acid | 10 | 3.17 ± 0.23 |
| MMP-2 | Hydroxamic acid | 37 | 0.83 ± 0.03 |
| MMP-9 | Peptide-amphiphile 1 | 10 | 1.76 ± 0.05 |
| MMP-9 | Peptide-amphiphile 1 | 37 | 1.29 ± 0.00 |
| MMP-9 | Peptide-amphiphile 2 | 10 | 2.20 ± 0.34 |
| MMP-9 | Peptide-amphiphile 2 | 37 | 2.34 ± 0.23 |

### Example 3

### Preparation of the Phosphonate Dipeptide Analog

Starting from the Fmoc-protected glycine phosphinate 5, the α-hydroxyacid 8 is coupled to it using water-soluble carbodiimide (EDC) and a catalytic amount of 4-dimethylaminopyridine (DMAP) to make the H-phosphinate ester 9. The α-hydroxyacids are readily available from the corresponding amino acids by diazotization in water. These can then be converted to the corresponding allyl esters by reaction with allylbromide under phase-transfer conditions. The carbodiimide-mediated coupling reaction for sterically unhindered phosphinates such as 5 is usually very high yielding. Next, using methods developed in the Hammer laboratory, (Rushing, S. D., and Hammer, R. P. (2001) Synthesis of phosphonamide and thiophosphonamide dipeptides, J. Am. Chem. Soc. 123, 4861-4862; Fernandez, M. d. F., Vlaar, C. P., Fan, H., Liu, Y.-H., Fronczek, F. R., and Hammer, R. P. (1995) Synthesis of phosphonate and thiophosphonate esters and amides from hydrogenphosphinates by a novel one-pot activation-coupling-oxidation procedure, J. Org. Chem. 60:7390-7391; Fernandez, M. d. F., Kappel, J. C., and Hammer, R. P. (1999) A new approach to phosphonopeptide analogs in Peptides: Chemistry. Structure and Biology (Proceedings of the Fifteenth American Peptide Symposium) (Tam, J. P., and Kaumaya, P. T. P., Eds.) pp 237-238, Kluwer, Dordrecht, The Netherlands) the H-phosphinate 9 can be nonoxidatively activated to the phosphonochloridite 10 with dichlorotriphenylphosphorane and then coupled with adamantyl alcohol and oxidized to give the fully protected phosphonate derivative, which upon Pd-catalyzed removal of the allyl ester gives the desired phosphonodipeptide 2. Alternatively, the H-phosphinate ester 9 could be oxidized by periodate and then esterified by the Ag₂O/Ad-Br method described above (Georgiadis, D., Matziari, M., and Yiotakis, A. (2001) A highly efficient method for the preparation of phosphinic pseudodipeptidic blocks suitably protected for solid-phase peptide synthesis, Tetrahedron 57, 3471-3478) to give fully blocked phosphono-dipeptide 11. This is again readily converted to desired free acid 2 by Pd-catalyzed removal of the allyl ester. The thiophosphonate can be prepared by sulfurizing compound 9 with sulfur under basic conditions and the resultant thiophosphinate can be alkylated with Ag₂O/Ad-Br as before. Deprotection of the allyl ester then gives the thiophosphinate protected derivative 2 suitable for solid-phase peptide synthesis.

The first inhibitor that has been prepared is modeled after the al (V)436-450 sequence. We previously utilized this sequence to create the potential substrate C₆-(Gly-Pro-Hyp)₄-Gly-Pro-Pro-Gly˜Val-Val-Gly-Glu-Gln-Gly-Glu-Gln-Gly-Pro-Pro-(Gly-Pro-Hyp)₄-NH₂ (SEQ ID NO: 5). MMP-1 and MMP-9 hydrolysis of this substrate was studied at 30° C. with 40 nM enzyme and 40 μM substrate. MMP-9 rapidly hydrolyzed the substrate within 1 h, while MMP-1 did not cleave the substrate even after 24 h. MALDI mass spectrometric analysis of the cleavage products indicated that the Gly-Val bond was cleaved by MMP-9. This is the analogous bond cleaved by MMP-9 in types V and XI collagen. No hydrolysis of the substrate by MMP-1 was detected using MALDI mass spectrometric analysis. The C₆-(Gly-Pro-Hyp)₄-Gly-Pro-Pro-Gly-Val-Val-Gly-Glu-Gln-Gly-Glu-Gln-Gly-Pro-Pro-(Gly-Pro-HYP)₄-NH₂ peptide-amphiphile (SEQ ID NO: 5) was the first substrate that shows complete selectivity between MMP-1 and MMP-9. (Lauer-Fields, J. L., Sritharan, T., Stack, M. S., Nagase, H., and Fields, G. B. (2003) Selective hydrolysis of triple-helical substrates by matrix metalloproteinase-2 and -9, J. Biol. Chem. 278:18140-18145).

For inhibitor design, the full α1 (V)436-450 sequence was incorporated with 5 Gly-Pro-Hyp repeats on both the N- and C-termini and a hexanoic acid coupled to the N-terminus. A phosphinate mimic site was used for the Gly˜Val residues. The sequence was purified by HPLC, analyzed by mass spectrometry, and then checked for triple-helicity using our standard circular dichroism spectroscopy protocols. (Yu, Y.-C., Tirrell, M., and Fields, G. B. (1998) Minimal lipidation stabilizes protein-like molecular architecture, J. Am. Chem. Soc. 120, 9979-9987; Lauer-Fjelds, J. L., Broder, T., Sritharan, T, Nagase, H., and Fields, G. B. (2001) Kinetic analysis of matrix metalloproteinase triple-helicase activity using fluorogenic substrates, Biochemistry 40:5795-580). The inhibitory constants (Kᵢ values) or half-inhibitory concentrations [IC₅₀ values] were determined for several MMPs. The Kᵢ values were 5.48 and 2.20 nM for MMP-2 and MMP-9, respectively, and 10-50 μM for MMP-8 and MMP-13 while no inhibition was seen for MMP-14, MMP-1 and MMP-3. Thus, the triple-helical peptide transition state analog was (a) an excellent inhibitor of MMP-2 and MMP-9 (the gelatinase members of the MMP family) and (b) a very poor inhibitor of all other MMPs tested. This relative selectivity between MMP family members has not been reported previously.

The second inhibitor that has been prepared is modeled after a collagenolytic MMP consensus cleavage site from types I-III collagen. We previously utilized this sequence to create the potential substrate (Gly-Pro-Hyp)₅-Gly-Pro-Gln-Gly˜Leu-Ala-Gly-Gln-Arg-Gly-Ile-Arg-(Gly-Pro-Hyp)₅-NH₂ (SEQ ID NO: 6). MMP-1, MMP-2, MMP-3, MMP-8, MMP-13, and MMP-14 hydrolysis of this substrate was studied at 30° C. with 40 nM enzyme and 40 μM substrate. All collagenolytic MMPs (MMP-1, MMP-2, MMP-8, MMP-13, and MMP-14) hydrolyzed the substrate efficiently, while MMP-3 did not. MALDI mass spectrometric analysis of the cleavage products indicated that the Gly-Leu bond was cleaved by collagenolytic MMPs. This is the analogous bond cleaved by collagenolytic MMPs in types I-III collagen. The (Gly-Pro-Hyp)₅-Gly-Pro-Gln-Gly˜Leu-Ala-Gly-Gln-Arg-Gly-Ile-Arg-(Gly-Pro-Hyp)₅-NH₂ (SEQ ID NO: 6) substrate shows selectivity between collagenolytic and non-collagenolytic MMPs. (Lauer-Fields, J. L., Broder, T., Sritharan, T., Chung, L., Nagase, H., and Fields, G. B. (2001) Kinetic Analysis of Matrix Metalloproteinase Activity Using Fluorogenic Triple-Helical Substrates, Biochemistry 40:5795-5803; Minond, D., Lauer-Fields, J. L., Nagase, H., and Fields, G. B. (2004) Matrix Metalloproteinase Triple-Helical Peptidase Activities are Differentially Regulated by Substrate Stability, Biochemistry 43:11474-11481; Hurst, D. R., Schwartz, M. A., Ghaffari, M. A., Jin, Y., Tschesche, H., Fields, G. B., and Sang, Q.-X. A (2004) Catalytic- and Ecto-domains of Membrane Type 1-Matrix Metalloproteinase have Similar Inhibition Profiles but Distinct Endopeptidase Activities, Biochem. J. 377:775-779; Minond, D., Lauer-Fields, J. L., Cudic, M., Overall, C. M., Pei, D., Brew, K., Visse, R., Nagase, H., and Fields, G. B. (2006) The Roles of Substrate Thermal Stability and P₂ and P₁′ Subsite Identity on Matrix Metalloproteinase Triple-Helical Peptidase Activity and Collagen Specificity, J. Biol. Chem. 281:in press (2006).

For inhibitor design, the full collagenolytic MMP consensus cleavage site from types I-III collagen was incorporated with 4 Gly-Pro-Hyp repeats and 1 Gly-Pro-Flp repeat on both the N- and C-termini. A phosphinate mimic site was used for the Gly˜Leu residues. The sequence was purified by HPLC, analyzed by mass spectrometry, and then checked for triple-helicity using our standard circular dichroism spectroscopy protocols. (Yu, Y.-C., Tirrell, M., and Fields, G. B. (1998) Minimal lipidation stabilizes protein-like molecular architecture, J. Am. Chem. Soc. 120, 9979-9987; Lauer-Fields, J. L., Broder, T., Sritharan, T, Nagase, H., and Fields, G. B. (2001) Kinetic analysis of matrix metalloproteinase triple-helicase activity using fluorogenic substrates, Biochemistry 40:5795-580). Kᵢ values were 18.6, 0.40, and 0.12 nM for MMP-1, MMP-2, and MMP-9, respectively. Thus, the second triple-helical peptide transition state analog was an excellent inhibitor of collagenolytic MMPs.

What is claimed is:

1. A pharmaceutical composition comprising a matrix metalloproteinase inhibitor wherein the inhibitor is a triple helix and/or poly-proline type II transition state analog phosphorus based inhibitor comprising a phosphonamide, phosphinic peptide or phosphonate ester.

2. The pharmaceutical composition of claim 1, wherein the phosphonamide is of the general formula: Ψ(PO₂H—NH).

3. The pharmaceutical composition of claim 1, wherein the phosphinic peptide is of the general formula: Ψ(PO₂H—CH₂).

4. The pharmaceutical composition of claim 1, wherein the phosphonate ester is of the general formula: Ψ{PO₂H—O}.

5. The pharmaceutical composition of claim 1, wherein the matrix metalloproteinase inhibitor comprises any one of SEQ ID NOS: 1-7.

6. The pharmaceutical composition of claim 1, wherein the inhibitor comprises at least one or more Gly-Pro-Hyp and Gly-Pro-Flp sequences.

7. The pharmaceutical composition of claim 1, wherein the inhibitor comprises at least one or more Gly-Pro-Hyp or Gly-Pro-Flp sequences.

8. The pharmaceutical composition of claim 6, wherein the Gly-Pro-Flp is at the N-terminus and/or C-terminus of the inhibitor.

9. The pharmaceutical composition of claim 1, wherein the inhibitor comprises a plurality of Gly-Pro-Hyp sequences.

10. The pharmaceutical composition of claim 1, wherein the inhibitor comprises between about one to ten Gly-Pro-Hyp sequences.

11. The pharmaceutical composition of claim 1, wherein inhibitor P and P′ subsites are substituted with molecules comprising phosphinate, phosphonate ester or phosphoramide mimics with Gly or Ala in the P₁ subsite and/or Cys(Mob) in the P₁′ subsite; Orn in the P₂ subsite; and, Glu in the P₂′ and/or P₃′ subsite.

12. A pharmaceutical composition comprising (R,S)-2-Isopropyl-3-((1-(N-(9-Fluorenylmethoxycarbonyl)amino)-methyl)-adamantyloxyphosphinyl)propanoic acid.

13. The pharmaceutical composition of claim 12, wherein the comprising substituted P, P₁, P₂′ and P₃′ subsites.

14. The pharmaceutical composition of claim 12, wherein the P and P′ subsites are substituted with molecules comprising phosphinate, phosphonate ester or phosphoramide mimics with Gly or Ala in the P₁ subsite and/or Cys(Mob) in the P₁′ subsite; Orn in the P₂ subsite; and, Glu in the P₂′ and/or P₃′ subsite.

15. A method of treating patients suffering from a matrix metalloproteinase mediated disease condition comprises:
administering to a patient in need thereof, a pharmaceutical composition comprising a matrix metalloproteinase inhibitor wherein the inhibitor is a triple helix phosphorus based inhibitor comprising a phosphonamide, phosphinic peptide or phosphonate ester.

16. The method of treating patients suffering from a matrix metalloproteinase mediated disease condition of claim 15, wherein the matrix metalloproteinase inhibitor comprises any one of SEQ ID NOS: 1-7.

17. The method of treating patients suffering from a matrix metalloproteinase mediated disease condition of claim 15, wherein the inhibitor comprises at least one or more Gly-Pro-Hyp and Gly-Pro-Flp sequences.

18. The method of treating patients suffering from a matrix metalloproteinase mediated disease condition of claim 15, wherein the inhibitor comprises at least one or more Gly-Pro-Hyp or Gly-Pro-Flp sequences.

19. The method of treating patients suffering from metalloproteinase mediated disease condition of claim 17, wherein the Gly-Pro-Flp is at the N-terminus and/or C-terminus of the inhibitor.
